SEQUENCE LISTING (75 sequences)

SEQ ID NO: 1 - protein, 14 aa, Artificial Sequence
  mitochondrial targeting sequence
GKPIPNPLLGLDST

SEQ ID NO: 2 - protein, 8 aa, Artificial Sequence
  nuclear localization sequence
DPKKKRKV

SEQ ID NO: 3 - protein, 12 aa, Artificial Sequence
  a nuclear export signal sequence
LQLPPLERLTLD

SEQ ID NO: 4 - protein, 21 aa, Artificial Sequence
  endoplasmic reticulum localization sequence
METDTLLLWVLLLWVPGSTGD

SEQ ID NO: 5 - protein, 27 aa, Artificial Sequence
  endoplasmic reticulum membrane targeting sequence
MDPVVVLGLCLSCLLLLSLWKQSYGGG

SEQ ID NO: 6 - DNA, 20 bp, Artificial Sequence
  MT-ND1 forward primer
cacctctagcctagccgttt

SEQ ID NO: 7 - DNA, 20 bp, Artificial Sequence
  MT-ND1 reverse primer
ccgatcagggcgtagtttga

SEQ ID NO: 8 - DNA, 21 bp, Artificial Sequence
  MT-ND2 forward primer
cttaaactccagcaccacgac

SEQ ID NO: 9 - DNA, 20 bp, Artificial Sequence
  MT-ND2 reverse primer
agcttgtttcaggtgcgaga

SEQ ID NO: 10 - DNA, 20 bp, Artificial Sequence
  MT-ND3 forward primer
ccgcgtccctttctccataa

SEQ ID NO: 11 - DNA, 20 bp, Artificial Sequence
  MT-ND3 reverse primer
agggctcatggtaggggtaa

SEQ ID NO: 12 - DNA, 20 bp, Artificial Sequence
  MT-ND4 forward primer
acaacacaatggggctcact

SEQ ID NO: 13 - DNA, 20 bp, Artificial Sequence
  MT-ND4 reverse primer
ccggtaatgatgtcggggtt

SEQ ID NO: 14 - DNA, 21 bp, Artificial Sequence
  MT-ND4L forward primer
tcgctcacacctcatatcctc

SEQ ID NO: 15 - DNA, 21 bp, Artificial Sequence
  MT-ND4L reverse primer
aggcggcaaagactagtatgg

SEQ ID NO: 16 - DNA, 20 bp, Artificial Sequence
  MT-ND5 forward primer
tccattgtcgcatccacctt

SEQ ID NO: 17 - DNA, 20 bp, Artificial Sequence
  MT-ND5 reverse primer
ggttgtttgggttgtggctc

SEQ ID NO: 18 - DNA, 20 bp, Artificial Sequence
  MT-ND6 forward primer
gggttgaggtcttggtgagt

SEQ ID NO: 19 - DNA, 20 bp, Artificial Sequence
  MT-ND6 reverse primer
accaatcctacctccatcgc

SEQ ID NO: 20 - DNA, 20 bp, Artificial Sequence
  MT-CYTB forward primer
tcttgcacgaaacgggatca

SEQ ID NO: 21 - DNA, 20 bp, Artificial Sequence
  MT-CYTB reverse primer
cgagggcgtctttgattgtg

SEQ ID NO: 22 - DNA, 20 bp, Artificial Sequence
  MT-COX1 forward primer
tccttattcgagccgagctg

SEQ ID NO: 23 - DNA, 20 bp, Artificial Sequence
  MT-COX1 reverse primer
acaaatgcatgggctgtgac

SEQ ID NO: 24 - DNA, 20 bp, Artificial Sequence
  MT-COX2 forward primer
aaccaaaccactttcaccgc

SEQ ID NO: 25 - DNA, 20 bp, Artificial Sequence
  MT-COX2 reverse primer
cgatgggcatgaaactgtgg

SEQ ID NO: 26 - DNA, 20 bp, Artificial Sequence
  MT-COX3 forward primer
ctaatgacctccggcctagc

SEQ ID NO: 27 - DNA, 20 bp, Artificial Sequence
  MT-COX3 reverse primer
aggcctagtatgaggagcgt

SEQ ID NO: 28 - DNA, 20 bp, Artificial Sequence
  MT-ATP6 forward primer
ttcgcttcattcattgcccc

SEQ ID NO: 29 - DNA, 21 bp, Artificial Sequence
  MT-ATP6 reverse primer
gggtggtgattagtcggttgt

SEQ ID NO: 30 - DNA, 21 bp, Artificial Sequence
  MT-ATP8 forward primer
actaccacctacctccctcac

SEQ ID NO: 31 - DNA, 22 bp, Artificial Sequence
  MT-ATP8 reverse primer
ggcaatgaatgaagcgaacaga

SEQ ID NO: 32 - DNA, 20 bp, Artificial Sequence
  MT-RNR1 forward primer
catccccgttccagtgagtt

SEQ ID NO: 33 - DNA, 21 bp, Artificial Sequence
  MT-RNR1 reverse primer
tggctaggctaagcgttttga

SEQ ID NO: 34 - DNA, 21 bp, Artificial Sequence
  MT-RNR2 forward primer
cagccgctattaaaggttcgt

SEQ ID NO: 35 - DNA, 20 bp, Artificial Sequence
  MT-RNR2 reverse primer
aaggcgctttgtgaagtagg

SEQ ID NO: 36 - DNA, 24 bp, Artificial Sequence
  GAPDH forward primer
ttcgacagtcagccgcatcttctt

SEQ ID NO: 37 - DNA, 24 bp, Artificial Sequence
  GAPDH reverse primer
gcccaatacgaccaaatccgttga

SEQ ID NO: 38 - DNA, 20 bp, Artificial Sequence
  XIST forward primer
ccctactagctcctcggaca

SEQ ID NO: 39 - DNA, 20 bp, Artificial Sequence
  XIST reverse primer
acacatgcagcgtggtatct

SEQ ID NO: 40 - DNA, 20 bp, Artificial Sequence
  EMC10 forward primer
ttcattgagcgcctggagat

SEQ ID NO: 41 - DNA, 20 bp, Artificial Sequence
  EMC10 reverse primer
ttcattgagcgcctggagat

SEQ ID NO: 42 - DNA, 18 bp, Artificial Sequence
  PCSK1N forward primer
gagacacccgacgtggac

SEQ ID NO: 43 - DNA, 20 bp, Artificial Sequence
  PCSK1N reverse primer
aatccgtcccagcaagtacc

SEQ ID NO: 44 - DNA, 21 bp, Artificial Sequence
  SSR2 forward primer
gtttgggatgccaacgatgag

SEQ ID NO: 45 - DNA, 20 bp, Artificial Sequence
  SSR2 reverse primer
ctccacggcgtatctgttca

SEQ ID NO: 46 - DNA, 20 bp, Artificial Sequence
  TMX1 forward primer
acggacgagaactggagaga

SEQ ID NO: 47 - DNA, 20 bp, Artificial Sequence
  TMX1 reverse primer
attttgacaagcagggcacc

SEQ ID NO: 48 - DNA, 21 bp, Artificial Sequence
  SFT2D2 forward primer
ccatcttcctcatgggaccag

SEQ ID NO: 49 - DNA, 20 bp, Artificial Sequence
  SFT2D2 reverse primer
gcagaacacagggtaagtgc

SEQ ID NO: 50 - DNA, 20 bp, Artificial Sequence
  EPT1 forward primer
tggctttctgctggtcgtat

SEQ ID NO: 51 - DNA, 20 bp, Artificial Sequence
  EPT1 reverse primer
aatccaaacccagtcaggca

SEQ ID NO: 52 - DNA, 20 bp, Artificial Sequence
  DRAP1 forward primer
acatcccacctgaagcagtg

SEQ ID NO: 53 - DNA, 20 bp, Artificial Sequence
  DRAP1 reverse primer
gatgccaccaggtccttcaa

SEQ ID NO: 54 - DNA, 20 bp, Artificial Sequence
  FAU forward primer
tcctaaggtggccaaacagg

SEQ ID NO: 55 - DNA, 20 bp, Artificial Sequence
  FAU reverse primer
gtgggcacaacgttgacaaa

SEQ ID NO: 56 - DNA, 20 bp, Artificial Sequence
  SUB1 forward primer
cgtcacttccggttctctgt

SEQ ID NO: 57 - DNA, 20 bp, Artificial Sequence
  SUB1 reverse primer
tgatttaggcatcgcttcgc

SEQ ID NO: 58 - DNA, 20 bp, Artificial Sequence
  LSM6 forward primer
cggacgaccagttgtggtaa

SEQ ID NO: 59 - DNA, 20 bp, Artificial Sequence
  LSM6 reverse primer
ccaggacccctcgataatcc

SEQ ID NO: 60 - DNA, 21 bp, Artificial Sequence
  COPS2 forward primer
aggaggactacgacctggaat

SEQ ID NO: 61 - DNA, 20 bp, Artificial Sequence
  COPS2 reverse primer
gccgcttttgggtcatcttc

SEQ ID NO: 62 - DNA, 20 bp, Artificial Sequence
  CGGBP1 forward primer
gcctcgtccactttccctaa

SEQ ID NO: 63 - DNA, 23 bp, Artificial Sequence
  CGGBP1 reverse primer
tcatgcctttacgtaggatcgag

SEQ ID NO: 64 - DNA, 20 bp, Artificial Sequence
  BCA53 forward primer
tcttgcctgctccacagttt

SEQ ID NO: 65 - DNA, 20 bp, Artificial Sequence
  BCA53 reverse primer
caaacaccaaggaggggtct

SEQ ID NO: 66 - DNA, 20 bp, Artificial Sequence
  CEP128 forward primer
tacagtaatggacaggcggg

SEQ ID NO: 67 - DNA, 20 bp, Artificial Sequence
  CEP128 reverse primer
tccggagttggtcgattgat

SEQ ID NO: 68 - DNA, 19 bp, Artificial Sequence
  MAD1L1 forward primer
cgagtctgccatcgtccaa

SEQ ID NO: 69 - DNA, 20 bp, Artificial Sequence
  MAD1L1 reverse primer
gcactctccacctgcttctt

SEQ ID NO: 70 - DNA, 19 bp, Artificial Sequence
  RAD51B forward primer
tttggacgaagccctgcat

SEQ ID NO: 71 - DNA, 20 bp, Artificial Sequence
  RAD51B reverse primer
cacaacctggtggacctgta

SEQ ID NO: 72 - DNA, 20 bp, Artificial Sequence
  RBPMS forward primer
acagtcgctcagaagcagag

SEQ ID NO: 73 - DNA, 20 bp, Artificial Sequence
  RBPMS reverse primer
cgaagcggatgccattcaaa

SEQ ID NO: 74 - DNA, 19 bp, Artificial Sequence
  TCF7 forward primer
tcaacagcccacatcccac

SEQ ID NO: 75 - DNA, 20 bp, Artificial Sequence
  TCF7 reverse primer
agaggcctgtgaacttgctt

(19) United States
(12) Patent Application Publication
(10) Pub. No.: US 2017/0227531 A1
(43) Pub. Date: Aug. 10, 2017

(54) METHODS FOR ISOLATING ENDOGENOUS NUCLEIC ACIDS FROM SUBCELLULAR COMPARTMENTS WITHOUT FRACTIONATION

(71) Applicants: The Board of Trustees of the Leland Stanford Junior University, Stanford, CA (US); Massachusetts Institute of Technology, Cambridge, MA (US); President and Fellows of Harvard College, Cambridge, MA (US)

(72) Inventors: Alice Ting, Stanford, CA (US); Pornchai Kaewsapsak, Stanford, CA (US); David M. Shechner, Arlington, MA (US); John L. Rinn, Cambridge, MA (US)

(21) Appl. No.: 15/424,823

(22) Filed: Feb. 4, 2017

Related U.S. Application Data
(60) Provisional application No. 62/291,214, filed on Feb. 4, 2016.

(51) Int. Cl. G01N 33/53; C12N 15/113; G01N 33/50; G01N 33/58; C12N 15/86; C12N 7/00

(52) U.S. Cl. CPC G01N 33/5308; C12N 15/86; C12N 7/00; G01N 33/5005; G01N 33/581; C12N 15/113; C12N 2740/15043

(57) ABSTRACT

Methods of determining subcellular localization of nucleic acids, including RNA and DNA are described. In particular, the invention relates to a method combining proximity-specific labeling with crosslinking of nucleic acids to proteins and sequencing to identify nucleic acids within or near a particular subcellular compartment in vivo.

### CROSS-REFERENCE TO RELATED APPLICATION

This application claims benefit under 35 U.S.C. §119(e) of provisional application 62/291,214, filed Feb. 4, 2016, which application is hereby incorporated by reference in its entirety.

### TECHNICAL FIELD

The present invention pertains generally to methods of determining subcellular localization of nucleic acids, including RNA and DNA. In particular, the invention relates to a method combining proximity-specific labeling with crosslinking of nucleic acids to proteins and sequencing to identify nucleic acids within or near a particular subcellular compartment in vivo.

### BACKGROUND

Ribonucleic acids (RNAs) comprise a diverse class of biomolecules that participate in a staggering breadth of fundamental processes in all living cells. Although, based on a small handful of examples, it has been speculated that subcellular localization may generally be a critical determinant of RNA function, current methods that identify the location of RNAs en masse have proven cumbersome, low-throughput, difficult and noisy. Most existing technologies for studying RNA localization are either based on microscopic fluorescence imaging, or require native purification of the target subcellular compartment in vitro. Methods in the former category are often extremely low-throughput (i.e. allowing only a handful of RNAs to be analyzed at a time), or alternatively require highly specialized next-generation microscopic equipment and/or a large array of custom biochemical reagents. Methods in the latter category require the development of a robust purification scheme for the target compartment, which may entail substantial loss of loosely affiliated RNAs, or may generally be impossible. In both cases, separating the biological signal from experimental noise can be extremely challenging.

Thus, there remains a need for a better, efficient, high-throughput methods of determining nucleic acid localization.

### SUMMARY

The present invention is based, in part, on the discovery of a new method for determining subcellular localization of nucleic acids, including RNA and DNA. In particular, the invention relates to a method combining proximity-specific labeling with crosslinking of nucleic acids to proteins and sequencing to identify nucleic acids within or near a particular subcellular compartment in vivo.

In one aspect, the invention includes method of mapping subcellular localization of nucleic acids in a cell, the method comprising: a) introducing a tagging enzyme into the cell, wherein the tagging enzyme is targeted to a subcellular region of interest; b) contacting the cell with a tagging substrate for the tagging enzyme, wherein the tagging enzyme catalyzes a reaction with the tagging substrate resulting in covalent attachment of a tag to proteins within an intracellular spatial location around the tagging enzyme; and c) contacting the cell with a crosslinking agent before or after step (b), wherein the crosslinking agent covalently couples the proteins to nearby nucleic acids to produce protein-nucleic acid fusions; d) isolating the tagged protein-nucleic acid fusions using an agent that selectively binds to the tag; and e) analyzing the tagged protein-nucleic acid fusions to produce a map of the subcellular localization of the nucleic acids.

Crosslinking of proteins and nucleic acids can be performed with any suitable crosslinking agent or technique known in the art. Exemplary crosslinking agents include formaldehyde, glutaraldehyde, dimethyl suberimidate, N-hydroxysuccinimide, and compounds comprising reactive groups, such as adiazomethane, diazoacetyl, or carbodiimide functional groups. Crosslinking can also be performed using click chemistry with suitable compounds comprising reactive azide or alkyne functional groups. For example, the tagging substrate can be a phenol derivative comprising an alkyne or azide functional group suitable for crosslinking by click chemistry. Alternatively, crosslinking can be performed using ultraviolet light.

In certain embodiments, the tagged protein-nucleic acid fusions are isolated using an agent, such as an antibody, a probe, a ligand, or an aptamer that selectively binds to the tag. The agent may be immobilized on a solid support, such as, but not limited to, a magnetic bead, latex bead, microtiter plate well, glass plate, nylon, agarose, or acrylamide. In another embodiment, the method further comprises lysing the cell.

In certain embodiments, the tagging enzyme is a peroxidase. Exemplary peroxidases include horseradish peroxidase and ascorbate peroxidase. In one embodiment, the tagging enzyme is an engineered ascorbate peroxidase (e.g., APEX or APEX2). Phenol and phenolic compounds such as tyramine or phenolic aryl azide derivatives react with hydrogen peroxide to generate short lived, reactive free radicals. For example, proximity labeling can be performed in the presence of hydrogen peroxide and biotin-phenol (BP), wherein the peroxidase catalyzes the reaction of the biotin-phenol with the hydrogen peroxide to produce a biotin-phenoxyl radical that reacts with nearby proteins resulting in biotinylation (i.e., tagging) of the proteins.

In other embodiments, the tagging enzyme is a biotin ligase. Exemplary biotin ligases include BirA and engineered variants thereof that nonspecifically biotinylate lysine residues of proteins. Biotin is provided to the cell as a substrate for the biotinylation reaction catalyzed by the biotin ligase.

Biotinylated protein-nucleic acid fusions, produced with either a peroxidase or biotin ligase, as described herein, can be isolated with a biotin-binding protein, such as streptavidin or avidin.

In another embodiment, the method further comprises treating the cell with a radical quencher (e.g., ascorbate or 6-hydroxy-2,5,7,8-tetramethylchroman-2-carboxylic acid (TROLOX)) after said tagging of the proteins.

In certain embodiments, the tagging enzyme comprises a targeting sequence that directs the tagging enzyme to the subcellular region of interest. Exemplary targeting sequences include a secretory protein signal sequence, a membrane protein signal sequence, a nuclear localization sequence, a mitochondrial localization sequence, an outer mitochondrial membrane sequence, an endoplasmic reticulum localization sequence, an endoplasmic reticulum membrane targeting sequence, a nucleolar localization signal sequence, a nuclear export signal sequence, a peroxisome localization sequence, and a protein binding motif sequence. In another embodiment, the targeting sequence comprises a sequence selected from the group consisting of SEQ ID NOS:1-5.

In other embodiments, the tagging enzyme is covalently linked to a peptide or protein that directs the tagging enzyme to the subcellular region of interest, such as a cytosolic protein, a nuclear protein, a membrane protein, a mitochondrial protein, a P-body protein, or a secretory pathway protein.

In another embodiment, introducing the tagging enzyme into the cell comprises transfecting the cell with a recombinant polynucleotide comprising a promoter operably linked to a polynucleotide encoding the tagging enzyme. The recombinant polynucleotide may comprise an expression vector, for example, a bacterial plasmid vector or a viral expression vector, such as, but not limited to, an adenovirus, retrovirus (e.g., γ-retrovirus and lentivirus), poxvirus, adeno-associated virus, baculovirus, or herpes simplex virus vector.

The cell can be any type of cell, including any eukaryotic cell, prokaryotic cell, or archaeon cell. For example, the cell may be an animal cell, plant cell, fungal cell, or protist cell. Alternatively, the cell can be an artificial cell, such as a nanoparticle, liposome, polymersome, or microcapsule encapsulating the nucleic acids.

RNA isolated and mapped by the methods described herein can be animal RNA, bacterial RNA, fungal RNA, protist RNA, or plant RNA. In one embodiment, the RNA is human RNA.

In another embodiment, the method further comprises amplifying at least one RNA or DNA molecules. RNA molecules may be amplified, for example, by performing reverse transcription polymerase chain reaction (RT-PCR).

In another embodiment, the method further comprises sequencing at least one RNA from the isolated tagged protein-RNA fusions.

In another embodiment, the method further comprises multiplex sequencing of the tagged protein-nucleic acid fusions. For example, sequencing may comprise performing deep sequencing or next-generation sequencing.

In another embodiment, the method further comprises identifying at least one RNA or DNA molecule in the tagged protein-nucleic acid fusions (e.g., of a messenger RNA, a ribosomal RNA, a transfer RNA, a non-coding RNA, and a regulatory RNA).

In another embodiment, the method further comprises identifying at least one ribonucleoprotein (RNP) interaction.

In another embodiment, the method further comprises calculating the frequencies of one or more RNA molecules that are present within the intracellular spatial location.

In another embodiment, the method further comprises quantitating one or more RNA molecules that are present within the intracellular spatial location.

In certain embodiments, the cell is exposed to a test condition prior to said contacting the cell with the tagging substrate or the crosslinking agent. For example, a test condition may comprise exposing the cell to a drug, a ligand for a receptor, a hormone, a second messenger, a pathogen, or a genetic modification. For example, the cell can be genetically modified by introducing a vector, short hairpin RNA (shRNA), small interfering RNA (siRNA), microRNA (miRNA), or CRISPR-associated system into the cell. Alternatively, a test condition may comprise exposing the cell to a change in temperature, growth media, membrane potential, or osmotic pressure.

In certain embodiments, a map of the subcellular localization of the RNA molecules, produced by the methods described herein, is compared to a reference map. For example, a map of the subcellular localization of the RNA molecules from a cell that is exposed to the test condition can be compared to a reference map of a cell that is not exposed to the test condition. In another embodiment, the method further comprises comparing a map of the subcellular localization of the nucleic acid molecules within the intracellular spatial location to a reference map for a cell at the same or a different developmental stage.

These and other embodiments of the subject invention will readily occur to those of skill in the art in view of the disclosure herein.

### BRIEF DESCRIPTION OF THE FIGURES

FIG. 1A-IF show development of APEX-RIP methodology and enrichment of mitochondrial RNAs from live cells. FIG. 1A shows a scheme showing optimized labeling protocol. Live HEK cells expressing APEX2 in the compartment of interest (in this example, the mitochondrial matrix) are treated with H₂O₂ in the presence of biotin-phenol for 1 minute (B=biotin), followed by 0.1% formaldehyde for 10 minutes to crosslink proteins to RNAs. Then cells are lysed, and biotinylated species enriched with streptavidin beads. RNAs are eluted and analyzed by qPCR or RNA Seq. IMM=inner mitochondrial membrane. FIG. 1B shows fluorescence imaging following the labeling scheme in (A). Anti-V5 antibody detects the mito-APEX2 construct, while neutravidin detects the species biotinylated by APEX2. The bottom row shows a negative control in which H₂O₂ was omitted from the BP labeling reaction. Scale bars, 10 μm. FIG. 1C shows streptavidin blot analysis of whole cell lysate after performing the labeling shown in (FIG. 1A). Lanes 2, 3, and 4 show negative controls in which APEX, H₂O₂, or BP were omitted. FIG. 1D shows RNA Seq results following the labeling protocol in (FIG. 1A). Each detected gene is plotted according to its RNA abundance before streptavidin enrichment (x axis) and after streptavidin enrichment (y axis). 15 RNAs are known to reside in the mitochondrial matrix (Mercer et al., 2011) (apart from tRNAs and rRNAs, which are depleted by our library preparation). The two nuclear genome-encoded RNAs, RPPH1 and RMRP, are below the diagonal because they are dual localized to mitochondria and the nucleus [Wang et al Cell 2010], and the latter pool is depleted by the streptavidin enrichment step. FIG. 1E shows an alternative presentation of RNA Seq data, in which each detected gene is plotted according to its abundance (post-streptavidin enrichment) in the experiment versus in a negative control (with APEX labeling suppressed via omission of H₂O₂). The nuclear-encoded RNAs RPPH1 and RMRP now appear above the diagonal, because only the mitochondrial pool of these RNAs contributes to the post-streptavidin enrichment signal. FIG. 1F shows a depiction of the human mitochondrial genome and the elements we enrich in our experiment (outside circle) versus in the negative control (middle circle). tRNAs and rRNAs are not enriched because our RNA Seq library preparation deliberately depletes them.

FIGS. 2A-2H show APEX-RIP mapping of the global nuclear-cytoplasmic RNA distribution. FIG. 2A shows fluorescence imaging of APEX2-NLS and APEX2-NES constructs. Anti-V5 and anti-FLAG antibodies detect the APEX2-NLS and APEX2-NES constructs respectively, while neutravidin detects the species biotinylated by APEX2. Row 2 and 4 show negative controls in which H₂O₂ was omitted from the BP labeling reaction. Row 5 shows a negative control where untransfected cells were used. Scale bars, 10 μm. FIG. 2B shows combined examination of NLS- and NES-APEX2 enrichments distinguishes nuclear and cytoplasmically localized RNAs. Fold changes were calculated relative to matched input samples (FPKM_{post enrichment}/FPKM_{pre enrichment}) for each experimental condition. Displayed are the median values of three replicates, for all genes with FPKM≧1.0. The top 1000 nuclear and cytosolic RNAs are selected from ENCODE data. Histogram plots, summarizing the separation of these RNA standards by each APEX2 construct, are projected along the axes of the scatter plot; fold changes are on the same scales as the corresponding axes of the scatter. FIG. 2C shows APEX-RIP recapitulates the established nuclear-cytoplasmic distribution of mRNAs and lncRNAs. Top: APEX2-NLS vs. ES scatter plots, as in (FIG. 2B), for all (left), lncRNAs (middle) and mRNAs (right). Data are the median of three replicates, plotted on the same scale. RNAs enriched equally by each construct lie along the dotted red (y=x) line; the distance of each data point to this line is defined as the nuclear preference score. Bottom: histogram plots of the nuclear preference score for each class of RNA. A significance threshold, calculated by ROC analysis (dotted line; FIG. 7D), is used to define predominantly nuclear (score>0.39) and cytoplasmic (score<0.39) species. FIG. 2D shows read density plots from APEX2-NLS and NES RIP samples, demonstrating examples of RNAs with stereotypical and atypical localization, respectively. For each gene, a common y-scale is used for all read tracks. The EEFIA1 mRNA (first row) is predominantly cytoplasmic, while the XIST lncRNA (second row) is nuclear. The Clorf63 mRNA (third row) is predominantly nuclear, while the SNHG5 lncRNA (forth row) is cytoplasmic. SnoRNAs encoded in the SNHG5 gene body are indicated as gray rectangles. FIG. 2E shows coverage analysis of lncRNAs in nuclear RNA data sets (APEX-RIP nuclear enriched RNAs vs ENCODE nuclear fractionation). Among 827 lncRNAs with FPKM>=1, APEX-RIP detected 71.7% of them, while fractionation detected 43.4%. FIG. 2F shows the abundance of detected ER-proximal RNAs in nuclear RNA data sets. Among 1260 ER proximal RNAs by ribosome profiling, nuclear-cytosolic score detected 116 RNAs (9.2%), while nuclear/cytosolic fractionation detected 158 RNAs. FIG. 2G shows contaminant analysis of ER proximal RNAs by ribosome profiling in two nuclear RNA sets. In 5467 APEX-RIP enriched nuclear RNAs, 116 RNAs are ER proximal RNAs (2.1%/o). In 3056 ENCODE nuclear fractionation RNAs, 156 RNAs are ER proximal RNAs (5.2%). FIG. 2H shows a Venn diagram showing overlap of our APEX2-NLS dataset and ENCODE nuclear fractionation. Both methods share 2469 RNAs.

FIGS. 3A-3L show APEX-RIP mapping of endogenous RNAs proximal to the ER membrane. FIG. 3A shows the labeling scheme. HRP, targeted to the ER with a KDEL sequence, biotinylates proteins in the ER lumen. B=biotin. 0.1% Formaldehyde crosslinks those proteins to RNAs across the ER membrane. FIG. 3B shows an alternative labeling scheme, with APEX2 displayed on the ER membrane (ERM) facing the cytosol. FIG. 3C shows a comparison of labeling specificities for schemes shown in (FIG. 3A) and (FIG. 3B). After biotinylation and crosslinking, RNAs were enriched with streptavidin and detected by qPCR. For reference, the far right panel shows the same experiment performed with APEX2 expressed throughout the cytosol. Target genes are RNAs previously shown to be enriched at the ER membrane (Jan et al., 2014). Off-target genes are cytosol and nucleus-localized RNAs. FIG. 3D shows imaging of biotinylation catalyzed by HRP-KDEL in cells. HEK stably expressing HRP-KDEL were labeled with BP and crosslinked as in (FIG. 3A), then fixed with 4% formaldehyde, and stained with neutravidin-AlexaFlour647 conjugate to detect biotinylated species. The HRP construct was detected by anti-V5 staining. Bottom row is a negative control that omits H₂O₂ during the BP labeling step. DIC, Differential Interference Contrast. Scale bars, 10 μm. FIG. 3E shows streptavidin blot detection of endogenous proteins biotinylated by HRP-KDEL. HEK were labeled as in (FIG. 3D), then lysed and run on SDS-PAGE. Lane 2 is a negative control with H₂O₂ omitted. Arrowheads point to endogenously biotinylated proteins [Chapman-Smith, A et al J Nutr 1999]. FIG. 3F shows a scatter plot showing RNA abundance (FKPM) before versus after streptavidin enrichment. Known secretory mRNAs (dark gray) are enriched relative to non-secretory mRNAs (light gray). FIG. 3G shows histograms showing the distribution of post/pre-enrichment RNA abundance ratios (log₂(FKPM post-enrichment/FKPM pre-enrichment)) for mRNAs encoding known secretory proteins (top histogram, dark gray), and mRNAs encoding non-secretory proteins (bottom histogram, light gray). ROC analysis (FIG. 7D) was used to determine the cutoff ratio for our final ER-associated RNA list. FIG. 3H shows classification of RNAs in our ER-associated RNA list. Putative non-coding RNAs constitute 11.3% of the list. FIG. 3I shows specificity analysis for protein-coding mRNAs in our ER-associated RNA list. Of the 2635 mRNAs we enriched, 95% have secretory annotation, according to (in rank order) Phobius [Kall et al JMB 2004], TMHMM (which predicts transmembrane domains), SignalP (which predicts ER signal sequences), or Gene Ontology Cell Component (GOCC). For comparison, an identical analysis is performed for all human mRNAs, ER fractionation [Reid and Nicchitta 2012], and our ER-associated RNAs that are not part of ER proximal RNAs (Jan et all 2014). FIG. 3J shows a comparison of coverage of 71 ER protein-coding mRNAs. Of these 71 mRNAs, we detected 69 RNAs, while ER proximal ribosome profiling (Jan et al. 2014) and ER fractionation (Reid and Nicchitta 2012) detected only 55 and 52 RNAs respectively. FIG. 3K shows the overlap of ER-enriched RNA datasets Top: our ER enriched RNAs vs. ER fractionation. Both methods share 766 mRNAs. Bottom: our ER enriched RNas vs. ER proximal RNAs by ribosome profiling. These two methods share 1151 mRNAs. FIG. 3L shows RNA abundance of ER-associated RNAs from different methods. Dark gray bars represent RNAs enriched by HRP-KDEL RIP. The light gray bars represent RNAs from ER fractionation. The medium gray bars represent RNAs enriched by HRP-KDEL RIP but not by fractionation.

FIGS. 4A-4C show a further analysis of subcellular transcriptome. FIG. 4A shows a histogram of mitochondrial protein-coding mRNAs in ER membrane associated RNAs list by HRP-KDEL RIP. The RNA coding for mitochondrial proteins are distributed according to Log₂ FPKM ratio from HRP-KDEL RIP experiment. Dark gray represents the portion of genes that their encoded proteins predicted to have transmembrane domain by TMHMM. Light gray indicates no prediction of transmembrane domain. The dotted line denotes the cutoff from ROC analysis of secretory RNAs vs non secretory RNAs. FIG. 4B shows the classification of mitochondrial protein-coding mRNAs in our ER-associated RNA list (column one) for outer mitochondrial membrane proteins (OMM), inter mitochondrial space proteins (IMS), inner mitochondrial membrane proteins (IMM), and mitochondrial matrix proteins (matrix). In case of multiple location proteins, the priority ranks from OMM, IMS, IMM, then matrix. For comparison, the cytosolic RNA list by APEX2-NES/NLS and all annotated mitochondrial proteins were subjected to the same analysis and shown in column 2 and 3. FIG. 4C shows overlap between RNAs enriched by HRP-KDEL RIP and nuclear RNAs by Nuclear-Cyto score. Considering 5467 nuclear RNAs and 2970 ER-associated RNAs, there are 673 overlapping RNAs. For lncRNAs with FPKM>=1, 593 nuclear lncRNAs and 74 ER associated lncRNAs have 34 lncRNAs in common. This suggests 34 potential lncRNAs localized at nuclear lamina and 40 potential lncRNAs localized in cytoplasm.

FIGS. 5A-5D show additional RT-qPCR and RNA-seq analysis of mitochondrial RNAs. FIG. 5A shows a Western blot analysis of different APEX2 fusion constructs. HEK293T cells stably expressing mitochondrial matrix localized APEX2 (mito), nuclear-localized APEX2 (Nuc), cytoplasmic-localized APEX2 (Cyt), ER lumen-localized HRP (ER lumen), ER membrane facing cytosol APEX2 (ER mem), or none are biotin-phenol labeled and formaldehyde crosslinked. After labeling, the cells were analyzed by western blot. The left blot is ponceau stained to show protein loading. The top right blot is probed with streptavidin-HRP to detect biotinylation from different APEX constructs. The bottom blots are probed with anti V5 or anti FLAG antibodies to show expression of peroxidase constructs. FIG. 5B shows an initial RT-qPCR of mitochondrial RNA enrichment by mito-APEX2. Two protocols, in which the order of the biotin-phenol and formaldehyde crosslinking steps were reversed, were compared. HEK293T cells were transfected with either mito-APEX or mito-GFP. After BP labeling, HEK cells were lysed. The biotinylated proteins were enriched and the RNAs were reversed crosslinked. After cleaned-up by Agencourt RNAClean XP, the enriched RNA were reverse transcribed with random oligonucleotides and quantified by qPCR. RT-qPCR analysis shows the relative abundance of 15 mitochondrial RNAs (ATP6-8, CO1-3, CYB, ND1-6, and MTRNR1-2) compared to cytosolic (i.e., GAPDH) and nuclear (e.g., XIST) off-target RNAs (grey). The crosslinking first protocol, was used to generate the first mitochondrial transcriptome. FIG. 5C shows RT-qPCR analysis of mitochondrial RNA enrichment by mito-APEX2. Two protocols, I and II, were compared similarly to (FIG. 5B). RT-qPCR analysis shows the relative abundance of mitochondrial RNAs (dark blue) compared to cytosolic (e.g., GAPDH, HOOK2, and MAN2C1) and nuclear (e.g., XIST) off-target RNAs (grey). The second protocol, II, was used for all data shown in FIG. 1. FIG. 5D shows additional RNA Seq data from mitochondrial matrix APEX labeling experiment. Experimental replicates (replicates 1 and 2) and negative controls (right). Data points are colored according to mitochondrial (dark gray) versus non-mitochondrial (light gray) annotation.

FIGS. 6A-6F show analysis of ENCODE nuclear-cytosolic fractionation and RNA-seq data from APEX2-NLS and APEX2-NES RIP. FIG. 6A shows a scatter plot of RNAs from ENCODE nuclear-cytosolic fractionation. X axis is log base 2 of the ratio between RNA abundance (in FPKM) of nuclear fractionation over whole cell transcriptome. Y axis is log base 2 of the ratio between RNA abundance (in FPKM) of cytosolic fractionation over whole cell transcriptome. FIG. 6B shows histogram analysis of data in (FIG. 6A) ranked by nuclear preference, which is defined by the distance of each RNAs from Y═X line in (FIG. 6A). Long noncoding RNAs (lncRNAs) are colored in green and mRNAs are colored in red. FIG. 6C shows ROC analysis of nuclear-cytosolic RNAs by fractionation. For each nuclear preference cutoff, the True Positive Rate (TPR) was plotted against the False Positive Rate (FPR). TPR is defined as the fraction of lncRNAs above the cutoff. FPR is defined as the fraction of mRNAs above the cutoff. FIG. 6D shows TPR-FPR values are plotted for each nuclear preference. The first local maximum with highest nuclear preference was chosen to obtain the final nuclear RNA list by fractionation. FIG. 6E shows scatter plots of RNA-seq data (replicate 1-3) from APEX2-NLS. Each detected gene is plotted according to its RNA abundance before streptavidin enrichment (x axis) and after streptavidin enrichment (y axis). The top 1000 nuclear and cytosolic RNAs by ENCODE are colored in dark gray and light gray respectively. Other RNAs are colored in black. FIG. 6F shows scatter plots of RNA-seq data (replicate 1-3) from APEX2-NES.

FIGS. 7A-7E show qRT-PCR and RNA-seq analysis of ER associated RNAs. FIG. 7A shows an introduction of a radical quenching step between APEX2 labeling and formaldehyde crosslinking improves the specificity of RNA capture. After HRP-KDEL expressing cells were BP-labeled with 1 minutes H₂O₂, the cells quenched with or without the presence of 0.1% formaldehyde for 1 minutes. Then they were further crosslinked with formaldehyde to complete total time of 10 minutes. The enriched RNAs were obtained as the protocol mitoAPEX2 case and quantified the percent yield by qPCR. The dark gray bars represent ER-associated RNAs. The first three grey bars are the cytosolic RNAs that has relative low percent yield in FIG. 3. The last six grey bars are the cytosolic RNAs we identified after the first RNA-seq (data not shown) and we used them to optimize for the new condition. FIGS. 7B and 7C show scatter plots and histogram analysis of ER-associated RNAs by HRP-KDEL Same as analyses shown in FIGS. 3F and 3G, but for Replicate 2 (FIG. 7B) and Replicate 3 (FIG. 7C). Because the data quality was not as good for Replicate 3 (much poorer separation of true positives from false positives, by FPKM post/pre-enrichment ratio), we did not use Replicate 3 data for the determination of our final ER-associated RNA list. FIG. 7D shows a ROC analysis of ER datasets. For each FPKM ratio cutoff, the True Positive Rate (TPR) was plotted against the False Positive Rate (FPR). TPR is defined as the fraction of mRNAs with secretory annotation above the cutoff. FPR is defined as the fraction of mRNAs lacking secretory annotation above the cutoff. On the bottom, TPR-FPR values are plotted for each FKPM ratio. The maxima of the Replicate 1 and Replicate 2 plots were applied as cutoffs, and the results intersected, to obtain the final ER-associated RNA list.

FIG. 7E shows gene ontology (GO) analysis of non-secretory annotated RNAs in ER fractionation and our ER enriched RNAs. The GO terms shown here are the term that have p-value<0.05.

### DETAILED DESCRIPTION

The practice of the present invention will employ, unless otherwise indicated, conventional methods of pharmacology, chemistry, biochemistry, recombinant DNA techniques and immunology, within the skill of the art. Such techniques are explained fully in the literature. See, e.g., A. L. Lehninger, Biochemistry (Worth Publishers, Inc., current addition); Sambrook, et al., Molecular Cloning: A Laboratory Manual (3^{rd} Edition, 2001); RNA: Methods and Protocols (Methods in Molecular Biology, edited by H. Nielsen, Humana Press, 1st edition, 2010); Rio et al. RNA: A Laboratory Manual (Cold Spring Harbor Laboratory Press; 1st edition, 2010); Farrell RNA Methodologies: Laboratory Guide for Isolation and Characterization (Academic Press; 4^{th} edition, 2009); Methods In Enzymology (S. Colowick and N. Kaplan eds., Academic Press, Inc.).

All publications, patents and patent applications cited herein, whether supra or infra, are hereby incorporated by reference in their entireties.

### I. Definitions

In describing the present invention, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “an RNA” includes a mixture of two or more RNA, and the like.

The term “about,” particularly in reference to a given quantity, is meant to encompass deviations of plus or minus five percent.

As used herein, a “cell” refers to any type of cell from a prokaryotic, eukaryotic, or archaeon organism, including bacteria, archaea, fungi, protists, plants, and animals, including cells from tissues, organs, and biopsies, as well as recombinant cells, cells from cell lines cultured in vitro, and cellular fragments, cell components, or organelles comprising nucleic acids. The term also encompasses artificial cells, such as nanoparticles, liposomes, polymersomes, or microcapsules encapsulating nucleic acids. A cell may include a fixed cell or a live cell. The methods described herein can be performed, for example, on a sample comprising a single cell or a population of cells.

A “live cell,” as used herein, refers to an intact cell, naturally occurring or modified. The live cell may be isolated from other cells, mixed with other cells in a culture, or within a tissue (partial or intact) or an organism. In some embodiments, the live cell is a cell engineered to express a tagging enzyme, for example, a peroxidase or biotin ligase. In some embodiments, the live cell expresses a tagging enzyme that is targeted to a subcellular compartment or structure, for example, via a localization signal within or fused to the tagging enzyme.

The terms “nucleic acid,” “nucleic acid molecule,” “polynucleotide,” and “oligonucleotide” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. There is no intended distinction in length between the terms “nucleic acid,” “nucleic acid molecule,” “polynucleotide,” and “oligonucleotide” and these terms will be used interchangeably.

The terms “protein,” “polypeptide,” and “peptide” refer to any compound comprising naturally occurring or synthetic amino acid polymers or amino acid-like molecules including but not limited to compounds comprising amino and/or imino molecules. No particular size is implied by use of the terms “protein,” “polypeptide,” and “peptide,” and these terms are used interchangeably.

The term “tagging enzyme” refers to an enzyme that catalyzes a reaction which leads to the conjugation of a tag to a set of molecules, for example, nucleic acids, proteins, carbohydrates, or lipids. In some embodiments, a tagging enzyme catalyzes a reaction that results in promiscuous labeling of molecules, e.g., proteins and/or nucleic acids in the vicinity of the enzyme.

The term “tagging substrate” refers to a substrate of a tagging enzyme that, during the tagging enzyme-catalyzed reaction, is converted into a reactive form (e.g., a radical or unstable intermediate with a reactive functional group), which reacts with and attaches to a molecule (e.g., a nucleic acid or protein) in the vicinity of the enzyme. In some embodiments, a reactive moiety of the tagging substrate attaches to a molecule by formation of a covalent bond between the tagging substrate and the molecule.

As used herein, the term “binding pair” refers to first and second molecules that specifically bind to each other, such as a ligand and a receptor, an antigen and an antibody, or biotin and streptavidin. “Specific binding” of the first member of the binding pair to the second member of the binding pair in a sample is evidenced by the binding of the first member to the second member, or vice versa, with greater affinity and specificity than to other components in the sample. The binding between the members of the binding pair is typically noncovalent.

As used herein, a “solid support” refers to a solid surface such as a magnetic bead, latex bead, microtiter plate well, glass plate, nylon, agarose, acrylamide, and the like.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The terms “fusion protein,” “fusion polypeptide,” or “fusion peptide” as used herein refer to a fusion comprising a tagging enzyme in combination with a protein of interest as part of a single continuous chain of amino acids, which chain does not occur in nature. The tagging enzyme and the protein of interest may be connected directly to each other by peptide bonds or may be separated by intervening amino acid sequences. The protein of interest may be, for example, a cytosolic protein, a nuclear protein, a membrane protein, a mitochondrial protein, a P-body protein, a secretory pathway protein, or any other protein, wherein mapping its location and/or identifying it binding partners and/or nearby nucleic acids in a cell is of interest. The fusion protein may also contain other sequences such as targeting or localization sequences and/or tag sequences.

By “fragment” is intended a molecule consisting of only a part of the intact full length sequence and structure. The fragment can include a C-terminal deletion an N-terminal deletion, and/or an internal deletion of the polypeptide. Active fragments of a particular protein or polypeptide will generally include at least about 5-14 contiguous amino acid residues of the full length molecule, but may include at least about 15-25 contiguous amino acid residues of the full length molecule, and can include at least about 20-50 or more contiguous amino acid residues of the full length molecule, or any integer between 5 amino acids and the full length sequence, provided that the fragment in question retains biological activity.

“Substantially purified” generally refers to isolation of a substance (compound, polynucleotide, protein, polypeptide, peptide composition) such that the substance comprises the majority percent of the sample in which it resides. Typically, in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a protein, polypeptide or peptide, that the indicated molecule is separate and discrete from the whole organism with which the molecule is found in nature or is present in the substantial absence of other biological macro molecules of the same type. The term “isolated” with respect to a nucleic acid is a nucleic acid molecule devoid, in whole or part, of sequences normally associated with it in nature; or a sequence, as it exists in nature, but having heterologous sequences in association therewith; or a molecule disassociated from the chromosome.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells,” “host cells,” “cells”, “cell lines,” “cell cultures,” and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence.

“Expression cassette” or “expression construct” refers to an assembly which is capable of directing the expression of the sequence(s) or gene(s) of interest. An expression cassette generally includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single stranded DNA (e.g., a M13 origin of replication), at least one multiple cloning site, and a “mammalian” origin of replication (e.g., a SV40 or adenovirus origin of replication).

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material, and includes uptake of peptide- or antibody-linked DNAs.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses.

### I. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention relates to the development of a novel method for determining the subcellular localization of nucleic acids. In particular, the method combines proximity-specific labeling of proteins with crosslinking of nucleic acids to the labeled proteins to identify nucleic acids within or near a particular subcellular compartment in vivo and for mapping protein-nucleic acid interactions within a cell.

The method typically comprises the following steps: a) introducing a tagging enzyme into the cell, wherein the tagging enzyme is targeted to a subcellular region of interest; b) contacting the cell with a tagging substrate for the tagging enzyme, wherein the tagging enzyme catalyzes a reaction with the tagging substrate resulting in covalent attachment of a tag to proteins within an intracellular spatial location around the tagging enzyme; and c) contacting the cell with a crosslinking agent before or after step (b), wherein the crosslinking agent covalently couples the proteins to nearby nucleic acids to produce protein-nucleic acid fusions; d) isolating the tagged protein-nucleic acid fusions using an agent that selectively binds to the tag; and e) analyzing the tagged protein-nucleic acid fusions to produce a map of the subcellular localization of the nucleic acids.

The method may be applied to cell samples comprising a single cell or a population of cells of interest and can be performed on any type of cell, including any cell from a prokaryotic, eukaryotic, or archaeon organism, including bacteria, archaea, fungi, protists, plants, and animals. Cells from tissues, organs, and biopsies, as well as recombinant cells, cells from cell lines cultured in vitro, and artificial cells (e.g., nanoparticles, liposomes, polymersomes, or microcapsules encapsulating nucleic acids) may all be used in the practice of the invention. The methods of the invention are also applicable for investigating nucleic acid localization in cellular fragments, cell components, or organelles comprising nucleic acids.

Although the methods for tagging and the related reagents, materials and compositions described herein are well suited for use in live cells and tissues, it should be appreciated that their use is not so limited, but that they can also be applied to fixed cells and tissues, for example, fixed cells and tissues obtained from a subject, e.g., in a clinical setting. The methods may also be applied to lysed cells.

In general, the methods and strategies for tagging cellular proteins employ a tagging enzyme. In some embodiments, the tagging enzyme catalyzes a reaction with a tagging substrate that generates a reactive unstable reagent (e.g., a radical or reaction intermediate with a reactive functional group) that is capable of covalently labeling nearby proteins. The half-life of the tagging reagent generated by the tagging enzyme determines how far the reagent can travel from its point of generation before reacting with a molecule. Accordingly, the half-life of the reagent determines its labeling radius. Because the enzyme generated reagent has a short half-life, only proteins in proximity to the tagging enzyme and the reactive reagent generated by the tagging enzyme (typically a few tens to hundreds of nanometers) are covalently modified (i.e., tagged).

The tagging enzyme can be introduced into a cell and contacted with a tagging substrate under conditions suitable for the tagging enzyme to convert the tagging substrate into a reactive form that can react with and attach to molecules in the vicinity of the tagging enzyme. The tagging enzyme may be delivered to the cell interior or exterior, depending on which region of the cell is being analyzed. In some embodiments, the tagging enzyme is delivered to the interior of the cell, and in some instances, to specific subcellular compartments. In some embodiments, the tagging enzyme is delivered to a tissue. The tagging enzyme may also be introduced into a cell by transfecting the cell with a recombinant polynucleotide comprising a promoter operably linked to a polynucleotide encoding the tagging enzyme. The recombinant polynucleotide may comprise an expression vector, for example, a bacterial plasmid vector or a viral expression vector, such as, but not limited to, an adenovirus, retrovirus (e.g., γ-retrovirus and lentivirus), poxvirus, adeno-associated virus, baculovirus, or herpes simplex virus vector.

In some embodiments, the tagging enzyme is engineered to improve its capability in proximity labeling. For example, the tagging enzyme can be engineered to be expressed and/or active only within a subcellular compartment or structure of interest. The tagging enzyme may also be engineered to comprise one or more mutations that enhance its catalytic activity with a tagging substrate in a subcellular compartment or structure of interest.

The tagging enzyme can be directed to a specific protein or cellular compartment of interest in a number of ways. For example, the tagging enzyme may be modified to include a targeting sequence that directs the tagging enzyme to the subcellular region of interest. Targeting sequences that can be used include, but are not limited to, a secretory protein signal sequence, a membrane protein signal sequence, a nuclear localization sequence, a mitochondrial localization sequence, an outer mitochondrial membrane sequence, an endoplasmic reticulum localization sequence, an endoplasmic reticulum membrane targeting sequence, a nucleolar localization signal sequence, a nuclear export signal sequence, a peroxisome localization sequence, and a protein binding motif sequence. Exemplary targeting sequences are shown in Table 1 and include sequences selected from the group consisting of SEQ ID NOS:1-5.

In other embodiments, the tagging enzyme is covalently linked to a peptide or protein that directs the tagging enzyme to a subcellular region of interest, such as a cytosolic protein, a nuclear protein, a membrane protein, a mitochondrial protein, a P-body protein, or a secretory pathway protein. Attachment to the protein of interest results in proximity labeling of proteins surrounding the protein of interest in the locations where it resides in the cell. Alternatively, the tagging enzyme can be covalently linked to an antibody that specifically binds a particular epitope found on certain proteins in a subcellular region of interest, which similarly allows proximity labeling of surrounding nearby proteins.

In some embodiments, the tagging enzyme is a peroxidase. Peroxidases catalyze the reaction of phenol and phenolic compounds such as tyramine or phenolic aryl azide derivatives with hydrogen peroxide to generate short-lived, reactive free radicals. For example, proximity labeling can be performed in the presence of hydrogen peroxide and biotin-phenol (BP) or a derivative thereof (e.g., O-acetylated biotin-phenol), wherein the peroxidase catalyzes the reaction of the biotin-phenol with the hydrogen peroxide to produce a biotin-phenoxyl radical that reacts with nearby proteins resulting in biotinylation (i.e., tagging) of the proteins. Exemplary peroxidases suitable for use as tagging enzymes include horseradish peroxidase, soybean peroxidase, and ascorbate peroxidase. In certain embodiments, the tagging enzyme is an engineered ascorbate peroxidase (e.g., APEX or APEX2). An advantage of using certain engineered ascorbate peroxidases is they can be expressed and active in a reducing cellular environment. For a description of APEX and APEX2 engineered ascorbate peroxidases, see. e.g., Martell et al. (2012) Nat. Biotechnol. 30:1143-1148, Lam et al. (2015) Nat. Methods 12:51-54, and U.S. Patent Application Publication No. US 2014/0186870; herein incorporated by reference in their entireties.

In other embodiments, the tagging enzyme is a biotin ligase capable of adding a biotin tag to a protein. Biotin ligase catalyzes the reaction of biotin with ATP to produce biotinoyl-5′-AMP as a reaction intermediate. Normally, this reaction intermediate is retained in the active site of the enzyme until the biotin group is transferred to a specific target protein. However, variant forms of biotin protein ligase such as BirA release this reaction intermediate from the active site such that it nonspecifically biotinylates any nearby protein with exposed lysine residues. Any such variant biotin protein ligase capable of promiscuously labeling proteins can be used in the practice of the invention.

Crosslinking of nucleic acids to the tagged cellular proteins allows identification of nucleic acids (e.g., RNA or DNA) in the vicinity of the tagged proteins. Furthermore, such crosslinking allows nucleic acids to be mapped to particular organelles, including subcompartments of organelles without subcellular fractionation. Crosslinking agents that can be used for crosslinking proteins and nucleic acids include, but are not limited to, dimethyl suberimidate, N-hydroxysuccinimide, formaldehyde, and glutaraldehyde. In addition, carboxyl-reactive chemical groups such as diazomethane, diazoacetyl, and carbodiimide can be included for crosslinking carboxylic acids to primary amines. In particular, the carbodiimide compounds, 1-ethyl-3-(-3-dimethylaminopropyl) carbodiimide hydrochloride (EDC) and N′,N′-dicyclohexyl carbodiimide (DCC) can be used for conjugation with carboxylic acids. In order to improve the efficiency of crosslinking reactions, N-hydroxysuccinimide (NHS) or a water-soluble analog (e.g., Sulfo-NHS) may be used in combination with a carbodiimide compound. The carbodiimide compound (e.g., EDC or DCC) couples NHS to carboxyl groups to form an NHS ester intermediate, which readily reacts with primary amines at physiological pH. In addition, ultraviolet light can be used for crosslinking proteins to nucleic acids. For a description of various crosslinking agents and techniques, see, e.g., Wong and Jameson Chemistry of Protein and Nucleic Acid Cross-Linking and Conjugation (CRC Press, 2^{nd} edition, 2011), Hermanson Bioconjugate Techniques (Academic Press, 3^{rd} edition, 2013), herein incorporated by reference in their entireties.

In certain embodiments, crosslinking of proteins and nucleic acids is performed using click chemistry. Crosslinking of proteins and nucleic acids using click chemistry can be performed with suitable crosslinking agents comprising reactive azide or alkyne functional groups. For example, a peroxidase tagging substrate can be a phenol derivative comprising an alkyne or azide functional group suitable for crosslinking by click chemistry. See, e.g., Kolb et al., 2004, Angew Chem Int Ed 40:3004-31; Evans, 2007, Aust J Chem 60:384-95; Millward et al. (2013) Integr Biol (Camb) 5(1):87-95), Lallana et al. (2012) Pharm Res 29(1):1-34, Gregoritza et al. (2015) Eur J Pharm Biopharm. 97(Pt B):438-453, Musumeci et al. (2015) Curr Med Chem. 22(17):2022-2050, McKay et al. (2014) Chem Biol 21(9):1075-1101, Ulrich et al. (2014) Chemistry 20(1):34-41, Pasini (2013) Molecules 18(8):9512-9530, and Wangler et al. (2010) Curr Med Chem. 17(11):1092-1116; herein incorporated by reference in their entireties.

In particular, crosslinking can be performed using strain-promoted azide-alkyne cycloaddition (SPAAC) click chemistry, a Cu-free variation of click chemistry that is generally biocompatible with cells. SPAAC utilizes a substituted cyclooctyne having an internal alkyne in a strained ring system. Ring strain together with electron-withdrawing substituents in the cyclooctyne promote a [3+2] dipolar cycloaddition with an azide functional group. SPAAC can be used for bioconjugation and crosslinking by attaching azide and cyclooctyne moieties to molecules. For a description of SPAAC, see, e.g., Baskin et al. (2007) Proc Natl Acad Sci USA 104(43):16793-16797, Agard et al. (2006) ACS Chem. Biol. 1: 644-648, Codelli et al. (2008) J. Am. Chem. Soc. 130:11486-11493, Gordon et al. (2012) J. Am. Chem. Soc. 134:9199-9208, Jiang et al. (2015) Soft Matter 11(30):6029-6036, Jang et al. (2012) Bioconjug Chem. 23(11):2256-2261, Ornelas et al. (2010) J Am Chem Soc. 132(11):3923-3931; herein incorporated by reference in their entireties.

Crosslinked biotinylated protein-nucleic acid fusions, produced as described herein, can be isolated with a biotin-binding protein, such as streptavidin or avidin. The biotin-binding protein may be immobilized on a solid support (e.g., streptavidin beads or magnetic beads) to facilitate removal from a liquid. The isolated protein-nucleic acid fusions can then be analyzed to identify nucleic acids and/or proteins by any appropriate method (e.g., mass spectrometry or immunoassays for identification of proteins and sequencing or polymerase chain reaction (PCR) with suitable primers for identification of nucleic acids). RNA may be reverse transcribed into cDNA with a reverse transcriptase prior to performing PCR (i.e., RT-PCR) and/or sequencing.

Any high-throughput technique for sequencing the nucleic acids can be used in the practice of the invention. Deep sequencing of nucleic acids can be used, for example, to improve sequence accuracy and for determining the frequency of RNA molecules in particular subcellular compartments or regions. DNA sequencing techniques include dideoxy sequencing reactions (Sanger method) using labeled terminators or primers and gel separation in slab or capillary, sequencing by synthesis using reversibly terminated labeled nucleotides, pyrosequencing, 454 sequencing, sequencing by synthesis using allele specific hybridization to a library of labeled clones followed by ligation, real time monitoring of the incorporation of labeled nucleotides during a polymerization step, polony sequencing, SOLID sequencing, and the like.

Certain high-throughput methods of sequencing comprise a step in which individual molecules are spatially isolated on a solid surface where they are sequenced in parallel. Such solid surfaces may include nonporous surfaces (such as in Solexa sequencing, e.g. Bentley et al, Nature, 456: 53-59 (2008) or Complete Genomics sequencing, e.g. Drmanac et al, Science, 327: 78-81 (2010)), arrays of wells, which may include bead- or particle-bound templates (such as with 454, e.g. Margulies et al, Nature, 437: 376-380 (2005) or Ion Torrent sequencing, U.S. patent publication 2010/0137143 or 2010/0304982), micromachined membranes (such as with SMRT sequencing, e.g. Eid et al, Science, 323: 133-138 (2009)), or bead arrays (as with SOLiD sequencing or polony sequencing, e.g. Kim et al, Science, 316: 1481-1414 (2007)). Such methods may comprise amplifying the isolated molecules either before or after they are spatially isolated on a solid surface. Prior amplification may comprise emulsion-based amplification, such as emulsion PCR, or rolling circle amplification.

Of particular interest is sequencing on the Illumina MiSeq, NextSeq, and HiSeq platforms, which use reversible-terminator sequencing by synthesis technology (see, e.g., Shen et al. (2012) BMC Bioinformatics 13:160; Junemann et al. (2013) Nat. Biotechnol. 31(4):294-296; Glenn (2011) Mol. Ecol. Resour. 11(5):759-769; Thudi et al. (2012) Brief Funct. Genomics 11(1):3-11; herein incorporated by reference).

As discussed above, tagging enzymes can be genetically targeted to a cellular region of interest to identify nucleic acids in the vicinity of tagged proteins within a specific subcellular compartment or region (e.g., the nucleus, endoplasmic reticulum, Golgi, mitochondria, mitochondria outer membrane, mitochondria inner membrane, mitochondria matrix space, chloroplasts, synaptic cleft, presynaptic membrane, postsynaptic membrane, dendritic spines, transport vesicles, regions of contact between mitochondria and endoplasmic reticulum, nuclear membrane, etc.) can be specifically tagged. In some embodiments, proteins within particular cell types (e.g., astrocytes, dendrocytes, stem cells, etc.) can be specifically tagged, for example, proteins within a specific cell type within a complex tissue, animal, or cell population. In some embodiments, proteins within particular macromolecular complexes (e.g., protein complexes such as ribosomes, replisome, transcription complex, spliceosome, DNA repair complex, fatty acid synthase, polyketide synthase, non-ribosomal peptide synthase, glutamate receptor signaling complex, neurexin-neuroligin signaling complex, etc.) can be tagged. In each context, the tagged protein-nucleic acid fusions can be analyzed (e.g., isolated and identified) to map protein-nucleic acid localization of specific cells, cellular compartments or regions, or macromolecular complexes of interest. This information can be used for research, diagnostic, therapeutic, and other applications.

For example, cells may be isolated from a patient, amplified or differentiated using IPS cell technology (induced pluripotent stem cell), contacted with a vector (e.g., a viral vector) that expresses a tagging enzyme, for example, a tagging enzyme fused to a localization signal effecting localization of the tagging enzyme in a specific subcellular compartment. Labeling and crosslinking can be performed in the living cells, as described herein, and the resulting tagged protein-nucleic acid fusions can be analyzed, for example, to identify patient specific information that can be useful to assist in diagnostic, prognostic, and/or therapeutic decisions, and in drug screening assays.

A tagging substrate is typically provided in an inert, stable, or non-reactive form, e.g., a form that does not readily react with other molecules in living cells. Once in contact with an active tagging enzyme, the tagging substrate is converted from its stable form into a short-lived reactive form, for e.g., via generation of a reactive moiety, such as a radical, on the tagging substrate by the tagging enzyme. Some tagging substrates are, accordingly, also referred to as radical precursors. The reactive form of the tagging substrate then reacts with and attaches to a molecule, e.g., a protein, in the vicinity of the tagging enzyme. Accordingly, in some embodiments, a tagging substrate comprises an inert or stable moiety that can be converted by the tagging enzyme into a reactive moiety. The reaction of the tagging substrate with a molecule, e.g., a protein in the vicinity of the tagging enzyme, results in the tagging, or labeling, of the molecule. Typically, a tagging substrate comprises a tag, which is a functional moiety or structure that can be used to detect, identify, or isolate a molecule comprising the tag, e.g., a protein that has been tagged by reacting with a tagging substrate. Suitable tags include, but are not limited to, for example, a detectable label, a binding agent, such as biotin, or a fluorescent probe, a click chemistry handle, an azide, alkyne, phosphine, trans-cyclooctene, or a tetrazine moiety. In some embodiments, the reaction of the reactive form of the tagging substrate with a molecule, e.g., a protein, may lead to changes in the molecule, e.g., oxygenation, that can be exploited for detecting and/or isolating the changed molecules. Non-limiting examples of such tagging substrates are chromophores, e.g., resorufin, malachite green, KillerRed, Ru(bpy)₃²⁺, and miniSOG, which can generate reactive oxygen species that oxidize molecules in the vicinity of the respective tagging enzyme. The oxidation can be used to isolate and/or identify the oxidized molecules. In some embodiments, the reactive form of the tagging substrate crosses cell membranes, while in other embodiments membranes are impermeable to the reactive form of the tagging substrate.

A tag may be, in some embodiments, a detectable label. In some embodiments, a tag may be a functional moiety or structure that can be used to detect, isolate, or identify molecules comprising the tag. A tag may also be created as a result of a reactive form of a tagging substrate reacting with a molecule, e.g., the creation of oxidative damage on a protein by a reactive oxygen species may be a tag. In some embodiments, the tag is a biotin-based tag and the tagging enzyme, e.g., a peroxidase, generates a reactive biotin moiety that binds to proteins within the vicinity of the tagging enzyme. In some embodiments, the biotin-based tags are biotin tyramide molecules. In some embodiments, the tagging substrate is a peroxidase substrate.

Additional suitable tagging substrates will be apparent to those of skill in the art, and the invention is not limited in this respect. In some embodiments, the tag is an alkyne tyramide and the peroxidase generates a reactive moiety that binds to proteins within the vicinity of the peroxidase. The alkyne subsequently can be modified, for example, by a click chemistry reaction to attach a tag (e.g., a biotin tag). The tag can then be used for further analysis (e.g., isolation and identification). It should be noted that the invention is not limited to alkyne tyramide, but that any functional group that can be chemoselectively derivatized can be used. Some examples are: azide or alkyne or phosphine, or trans-cyclooctene, or tetrazine, or cyclooctyne, or ketone, or hydrazide, or aldehyde, or hydrazine.

In some embodiments, a tagging substrate for a peroxidase, for example, a biotinylated phenol or tyramide, is administered to cells or tissue in vivo, and proteins that are located within the vicinity of the expressed peroxidase are tagged, i.e., the biotin tyramide is converted into a reactive form by the tagging enzyme, here the peroxidase, and the reactive form reacts with and attaches to proteins in the vicinity of the peroxidase, resulting in biotin-tagging of the respective proteins. In the presence of peroxide (e.g., H₂O₂), the peroxidase converts the substrate into a short-lived, reactive intermediate, for example, a reactive phenol or tyramide radical, that can form a covalent bond with a protein.

In some embodiments, the reactive intermediate, once created, reacts with (labels) proteins that are within the vicinity of the peroxidase enzyme molecule. The term “within the vicinity” refers to the spatial location around the enzyme and/or substrate that is labeled. In some instances it may refer to a region of the cell such as a sub-cellular region, a membrane or protein complex. Alternatively it can be defined in terms of distance from the enzyme or substrate or a region i.e., as a diameter, circumference or linear distance. For example, in some embodiments, a molecule within the vicinity of a tagging enzyme is a molecule that is positioned less than about 900 nm, less than about 800 nm, less than about 700 nm, less than about 600 nm, less than about 500 nm, less than about 400 nm, less than about 300 nm, less than about 200 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm away from the active site of the tagging enzyme. In some embodiments, proteins that are not within the vicinity of the enzyme are not exposed to the reactive intermediate and hence not labeled. In some embodiments, expression or targeting of the tagging enzyme to a subcellular compartment results in quantitative tagging of virtually all proteins within that compartment.

In addition, other non-peroxidase strategies for labeling, including the use of other enzymes, light-triggered labeling, and cascade reactions may be used. For example, KatG (a mycobacterial catalase-peroxidase enzyme), CueO (a multi-copper oxidase), and bilirubin oxidase are three suitable tagging enzymes. Like peroxidases, all of these enzymes convert stable small molecule substrates into short-lived reactive species. Their advantage, however, is that they utilize O₂, and not H₂O₂, to catalyze their respective reactions, which may be advantageous in embodiments involving cells, subcellular compartments, or structures that are sensitive to H₂O₂ toxicity. KatG from M. tuberculosis is believed to oxidize the anti-tuberculosis drug isoniazid (an aryl hydrazide) into an acyl radical, which then diffuses out of the KatG active site to label the NADH moiety of InhA reductase. CueO and bilirubin oxidase convert phenols into phenoxyl radicals at physiological pH. They also lack disulfides, and have solved crystal structures, which facilitates engineering.

Photo-oxidation reactions may also be used in the methods of the invention. Chromophores such as resorufin, malachite green, KillerRed, Ru(bpy)₃²⁺, and miniSOG can be used as tagging substrates, as they generate reactive oxygen species, which diffuse very short distances (40 Å for singlet oxygen and 15 Å for hydroxyl radical) before oxidizing cellular molecules and thereby damaging them. These chromophores are the basis of Chromophore Assisted Light Inactivation, or CALI, which has been applied to cellular proteins. Common products of oxidative damage to proteins are aldehydes and ketones, which provide a handle for selective protein pull-down by hydrazine- or hydroxylamine-biotin conjugates. If photo-oxidation is performed in the presence of reducing substrates, such as phenols or anilines (e.g., diaminobenzidine, used for electron microscopy), organic radicals will be generated, which can be exploited for covalent protein labeling. An advantage of this photo-oxidation approach compared to peroxidase-mediated labeling is the use of O₂ instead of H₂O₂. In addition, hydroxyl radicals generated in type I photo-oxidation (by chromophores such as malachite green) are much more reactive than peroxidase-generated aryloxyl radicals (BDE 119 versus 88 kcal/mol), which should lead to greater depth of coverage.

An additional type of tagging enzyme such as based on a cascade reaction for covalent labeling in cells can be used. Enediyne antibiotic prodrugs such as calicheamicin are activated inside cells to generate highly reactive 1,4-benzenoid diradicals. The structure of these prodrugs may be modified to make them activatable instead by orthogonal enzymes such as esterases or proteases, and, thus, useful as tagging substrates. N-nitrosoamides, which are converted by proteases via a cascade mechanism into reactive carbocations (with departure of N₂) may also be used as tagging substrates. Originally designed as protease suicide inhibitors, the carbocations were found to diffuse too rapidly from the site of generation and label neighboring molecules, making them particularly well suited for use as tagging substrates.

Thus, exemplary tagging enzymes include but are not limited to peroxidases, biotin ligases, KatG, CueO, and bilirubin oxidases. Exemplary tagging substrates include but are not limited to peroxidase substrates, such as phenols and tyramides, chromophores such as resorufin, malachite green, KillerRed, Ru(bpy)₃²⁺, and miniSOG, and enediyne antibiotic prodrugs such as calicheamicin.

In some embodiments, in vivo protein tagging is performed with a tagging enzyme that can be genetically targeted to any part of a live cell. In some embodiments, the tagging enzyme is present and/or active in all regions of the cell. In some embodiments, the tagging enzyme is present and/or active only in a subcellular compartment of the cell. In some embodiments, the tagging substrate is an exogenous small-molecule substrate that can be added or uncaged for the desired window of time, to permit precise temporal control of labeling. In some embodiments, the tagging substrate is conjugated to a binding agent, e.g., biotin (or other purification handle), for subsequent capture, e.g., by streptavidin-coated beads. In some embodiments, the tagging enzyme converts the substrate into a highly reactive species that has the potential to label any endogenous protein, in order to achieve high depth-of-coverage, e.g., in an MS experiment. In some embodiments, the reactive species has a short half-life on that its diffusion radius before quenching is less than approximately 100 nm, to ensure high specificity. In some embodiments, it is preferable for the reactive species not to cross cell membranes, to allow mapping of membrane-bounded structures.

In some embodiments, a tagging enzyme is engineered to be expressed and/or targeted in vivo or in situ to specific cells, cellular compartments (e.g., endoplasmic reticulum, Golgi apparatus, mitochondria, nucleus, the synaptic cleft, transport vesicles, etc.), and/or macromolecular complexes (e.g., protein complexes such as ribosomes, nuclear pore complex, fatty acid synthases) of interest. In some embodiments, a tagging enzyme is engineered to tag proteins that are located within a limited distance of the tagging enzyme. As a result, in some embodiments, proteins that are located within the targeted cell, cellular compartment, and/or macromolecular complex (e.g., protein complex) are specifically tagged relative to other proteins that are not located near the tagging enzyme. It should be appreciated that the tagging process itself does not need to be protein specific. For example, in some embodiments, it is the specific localization of the tagging enzyme that results in the specific tagging of a subset of proteins of interest. In some embodiments, proteins that are present within the vicinity of the tagging enzyme may be tagged for further analysis. In some embodiments, all proteins present within the vicinity of the tagging enzyme may be tagged. Various versions of the methodology offer a range of labeling radii, from about 500 nm to less than 10 nm, e.g., tagging radii of about 500 nm, about 400 nm, about 300 nm, about 250 nm, about 200 nm, about 100 nm, about 90 nm, about 80 nm, about 70 nm, about 60 nm, about 50 nm, about 40 nm, about 30 nm, about 20 nm, about 10 nm, about 5 nm, about 2.5 nm, or about 1 nm.

In some embodiments, the reactive moiety produced by the tagging enzyme, e.g., the peroxidase or biotin ligase, can be inactivated by contacting it with a quenching agent (e.g., water for an unstable reaction intermediate such as produced by biotin ligase, or a radical quencher such as ascorbate or 6-hydroxy-2,5,7,8-tetramethylchroman-2-carboxylic acid (TROLOX) after tagging with a peroxidase). As a result, the reactive moiety can have a short half-life and only modify proteins that are located within a short distance of the site of production (the peroxidase) before being inactivated. Accordingly, the zone of tagging can be limited by the diffusion rate of the reactive form of the tagging substrate, or the activated tagging moiety, and the half-life of the reactive form of the tagging substrate, or the activated tagging moiety.

In some embodiments, only proteins that are located within about 10 nm of the tagging enzyme are tagged. For example, in some embodiments using a peroxidase and a biotinylated peroxidase tagging substrate, e.g., a biotinylated phenol or tyramide, only proteins that are located within about 10 nm of the peroxidase are biotinylated. However, it should be appreciated that the zone of biotinylation may be altered depending on the enzyme and/or substrate structure used for tagging. Thus the labeling range can be adjusted from about 500 nm to <10 nm.

The methods provided herein can also be used to map nucleic acid localization in specific cell types within complex tissues or heterogeneous cell populations, or of specific subcellular structures or organelles within specific cells in complex tissues or populations. The methods are particularly useful for mapping subcellular localization of nucleic acids in rare cells within complex cell populations.

Maps of subcellular localization of nucleic acids can be developed not only for different cells, subcellular compartments, tissues, or organisms but also for cells, tissues, or organisms exposed to different conditions or environments. For example, cells or organisms exposed to different therapeutic agents, different concentrations of therapeutic agents, and/or combinations of therapeutic agents may be mapped and analyzed independently or compared against one another to examine changes occurring within a cell, tissue, or organism. Additionally, changes in nucleic acid localization in cells, tissues, or organisms over time associated with diseased states can be monitored by comparison of mapped nucleic acid localization in cells, tissues, or organisms in diseased and normal (i.e. healthy control, not having the disease) states.

In certain embodiments, a map of the subcellular localization of nucleic acids molecules, produced by the methods described herein, is compared to a reference map. For example, a map of the subcellular localization of the RNA molecules from a cell that is exposed to a test condition can be compared to a reference map of a cell that is not exposed to the test condition. A test condition may comprise, for example, exposing the cell to a drug, a ligand for a receptor, a hormone, a second messenger, a pathogen, or a genetic modification. For example, the cell can be genetically modified by introducing a vector, short hairpin RNA (shRNA), small interfering RNA (siRNA), microRNA (miRNA), or CRISPR-associated system into the cell. Alternatively, a test condition may comprise exposing the cell to a change in temperature, growth media, membrane potential, or osmotic pressure. In certain embodiments, the cell is exposed to a test condition prior to said contacting the cell with the tagging substrate or the crosslinking agent.

Maps of subcellular localization of nucleic acids can also be developed for cells, subcellular compartments, tissues, or organisms at different developmental stages. For example, a map of the subcellular localization of nucleic acids can be compared to reference maps for cells, subcellular compartments, tissues, or organisms at the same or different developmental stages.

### III. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### General Overview of Method for Isolating Endogenous RNAs from Subcellular Compartments without Fractionation

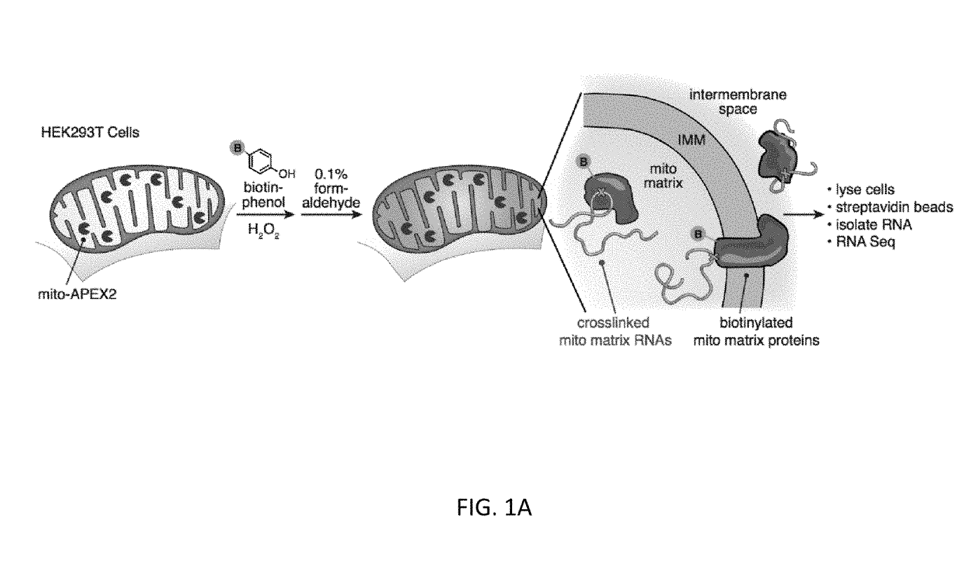

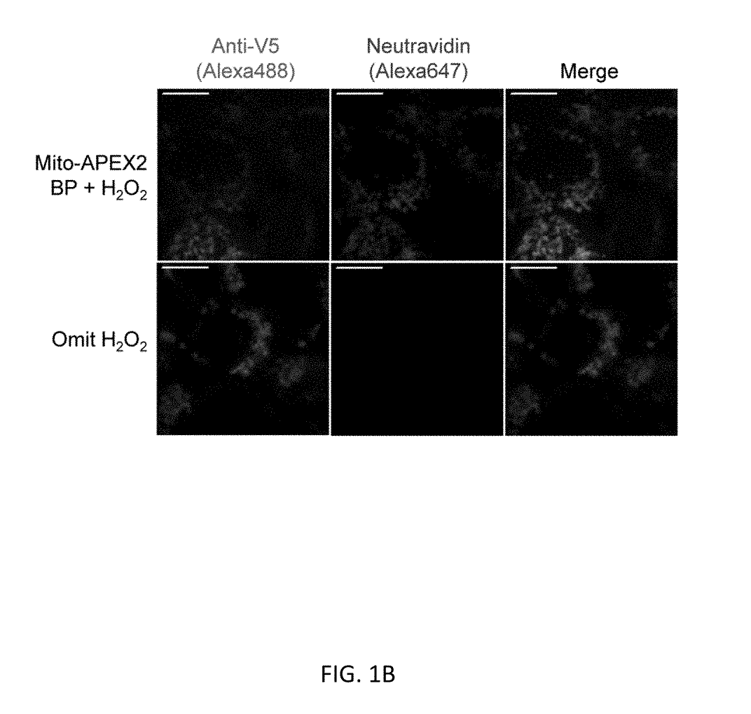

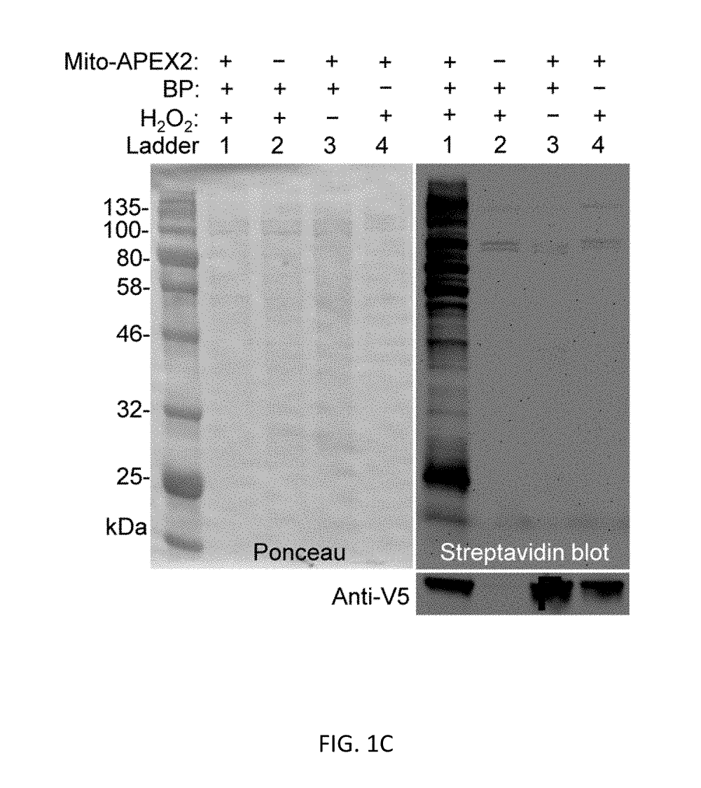

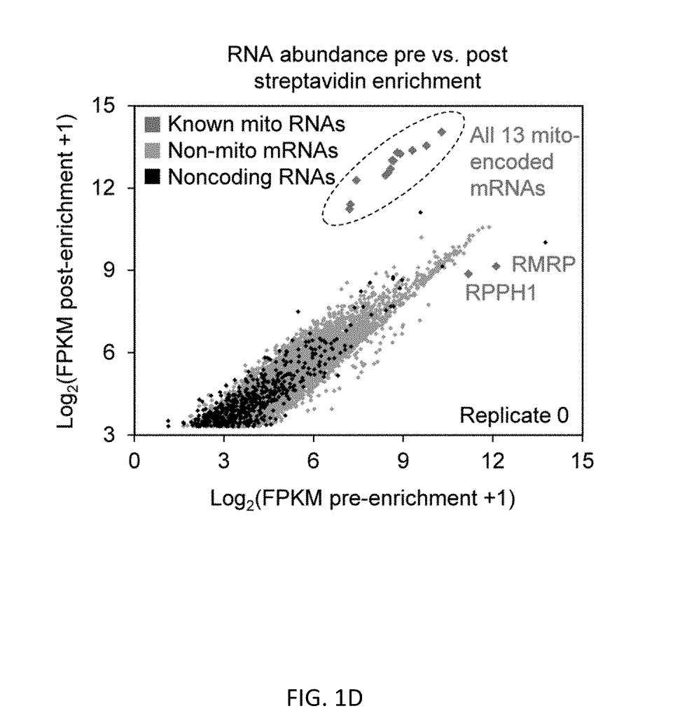

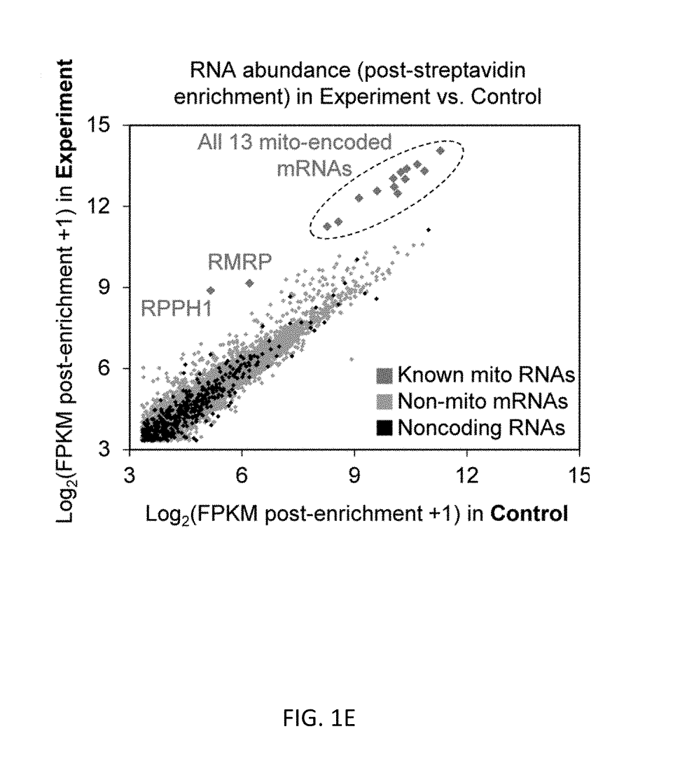

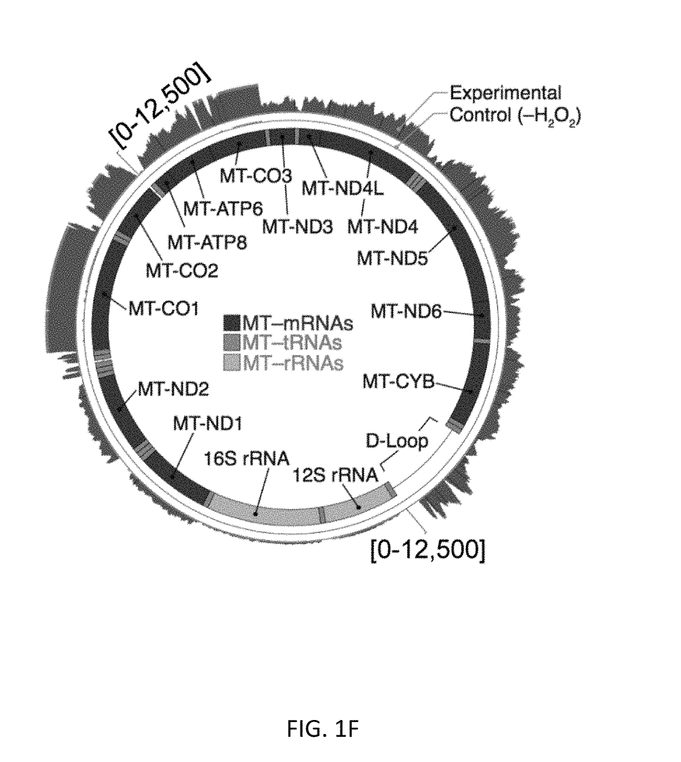

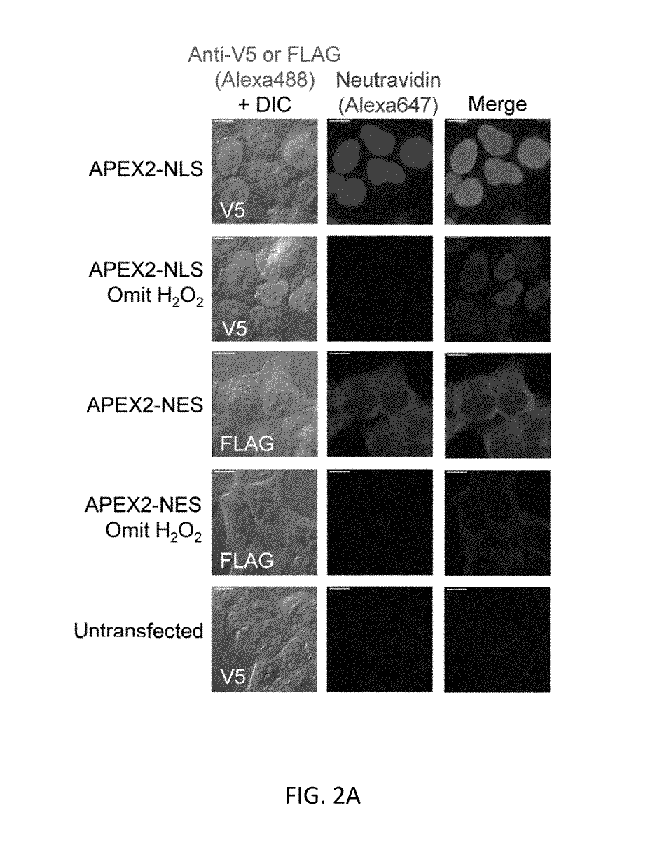

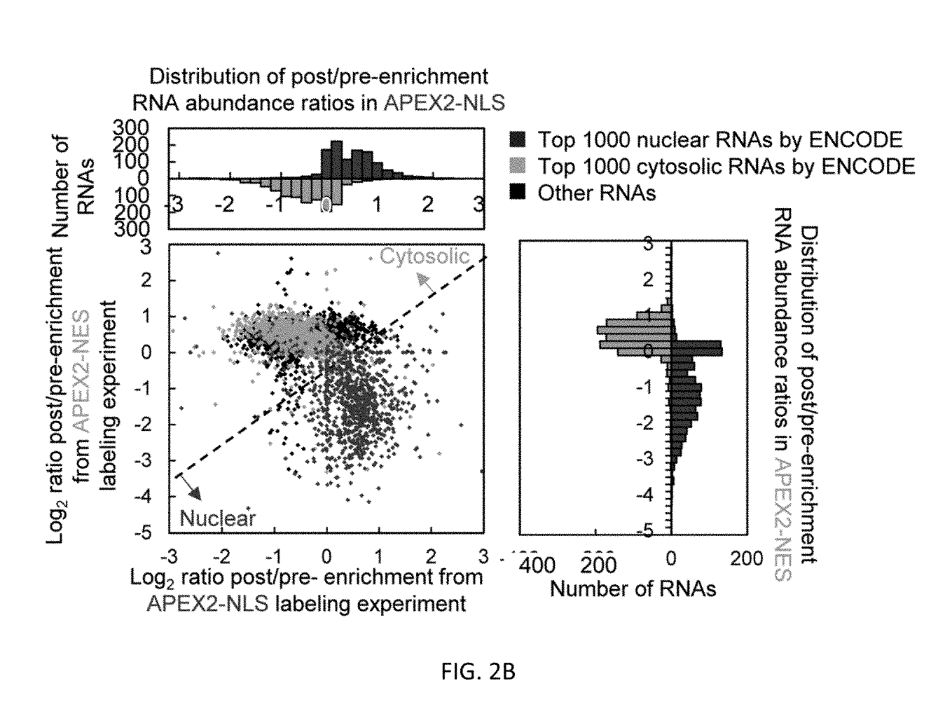

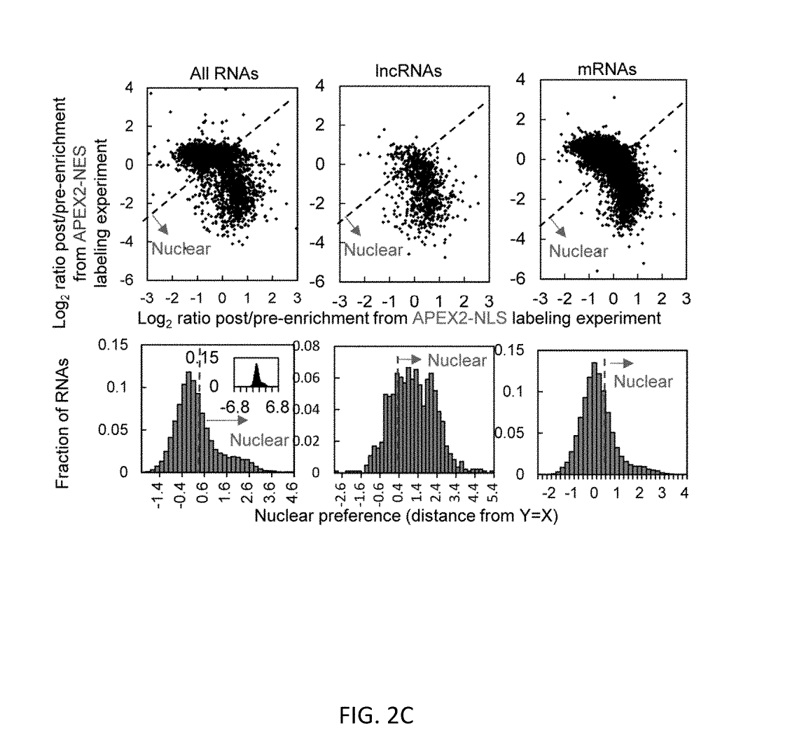

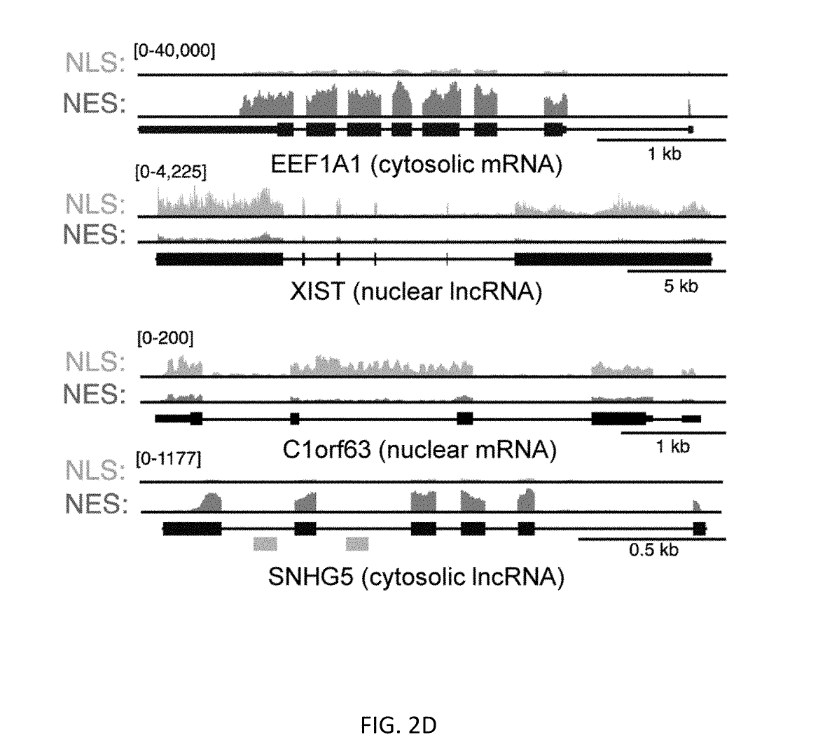

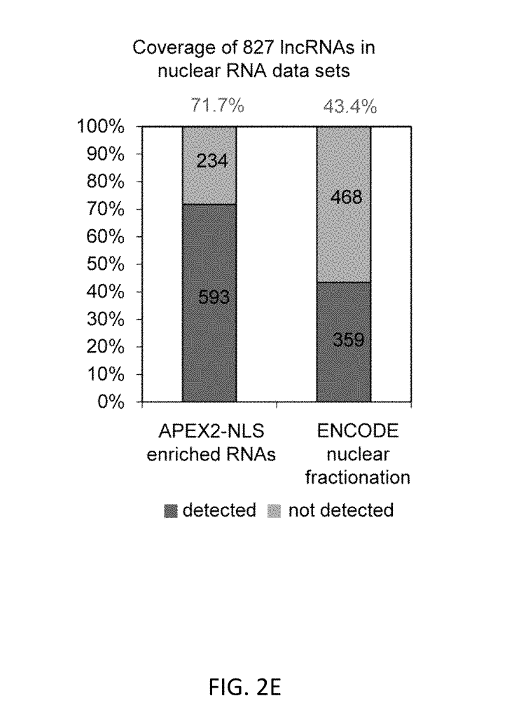

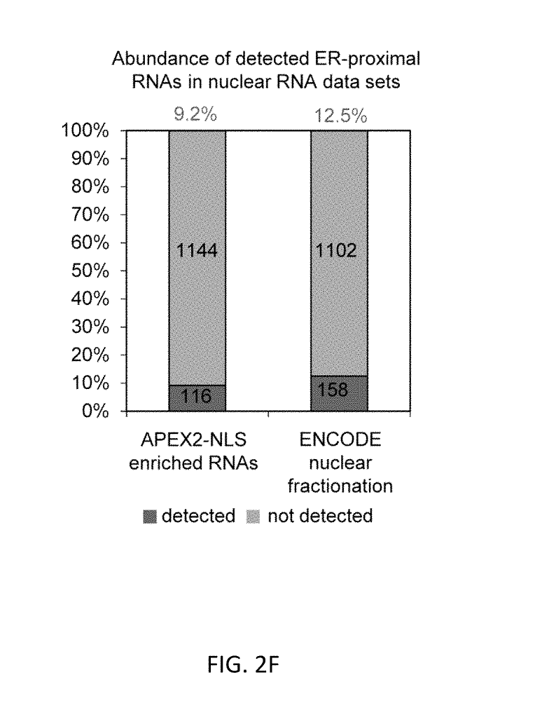

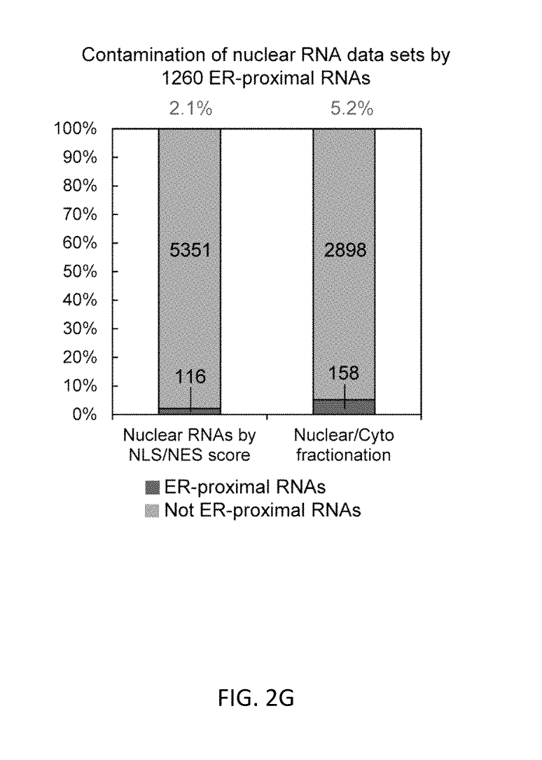

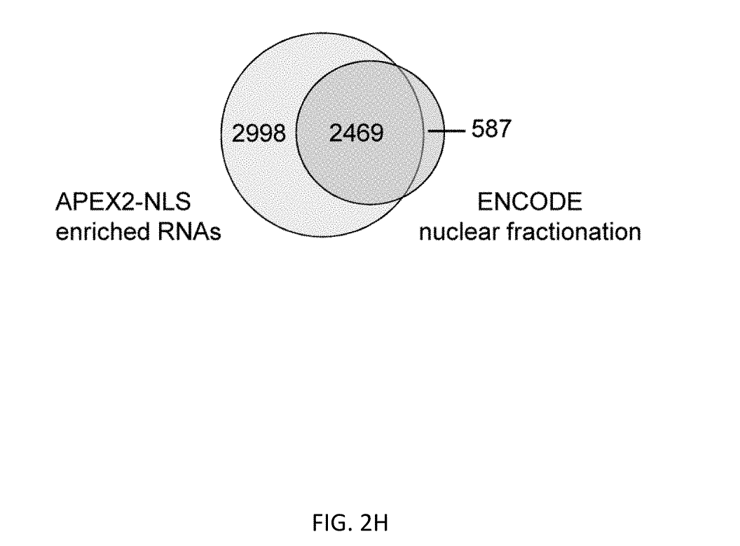

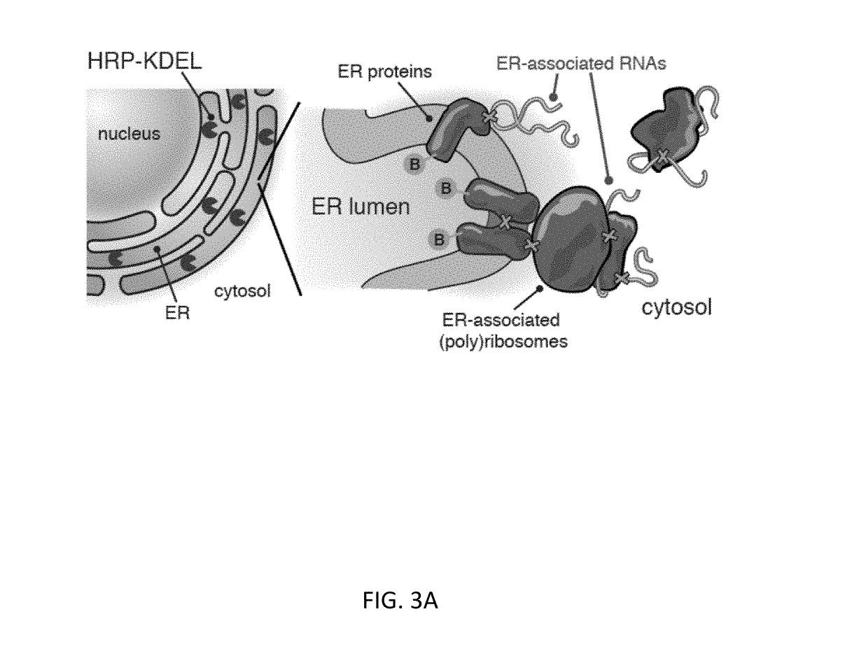

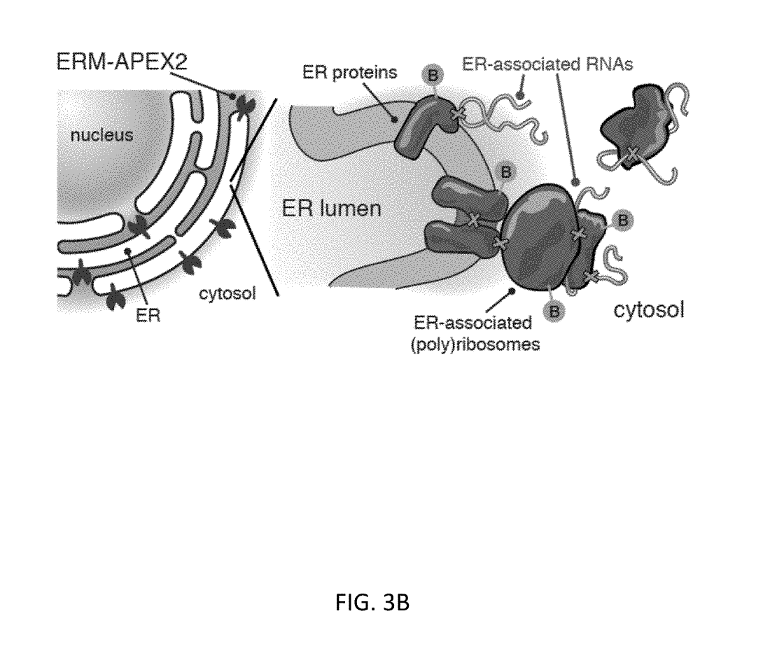

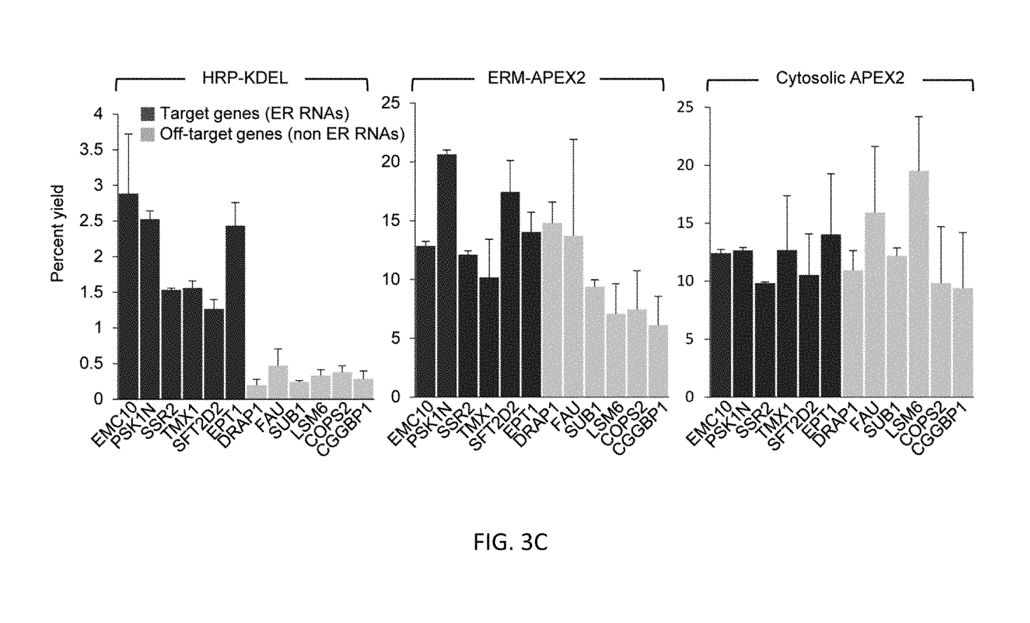

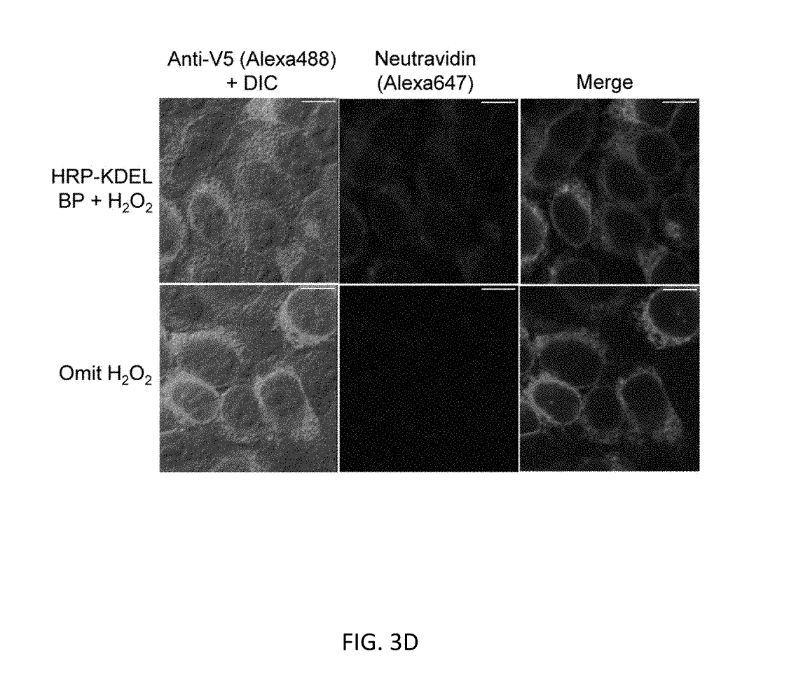

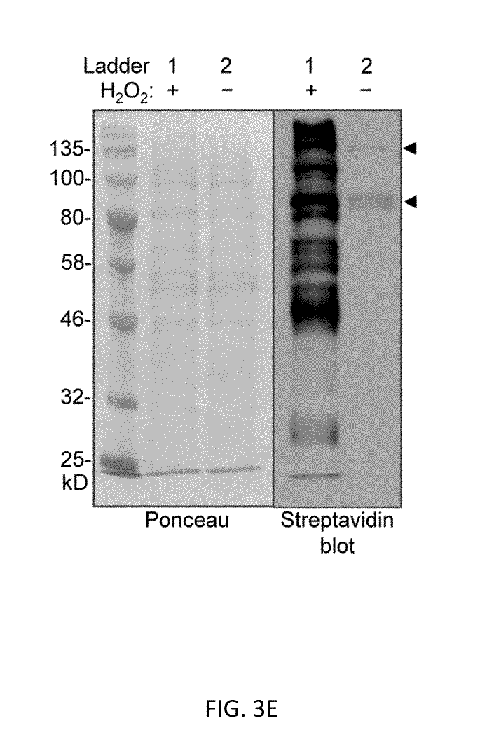

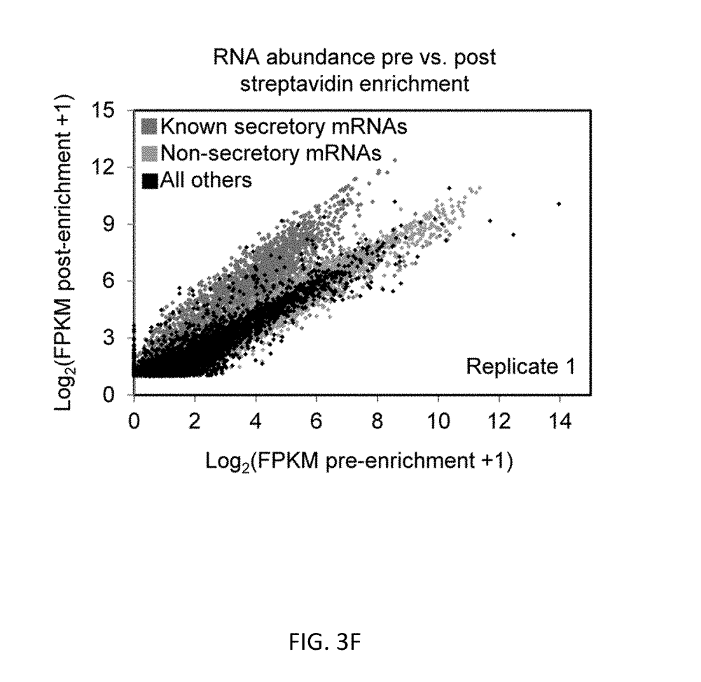

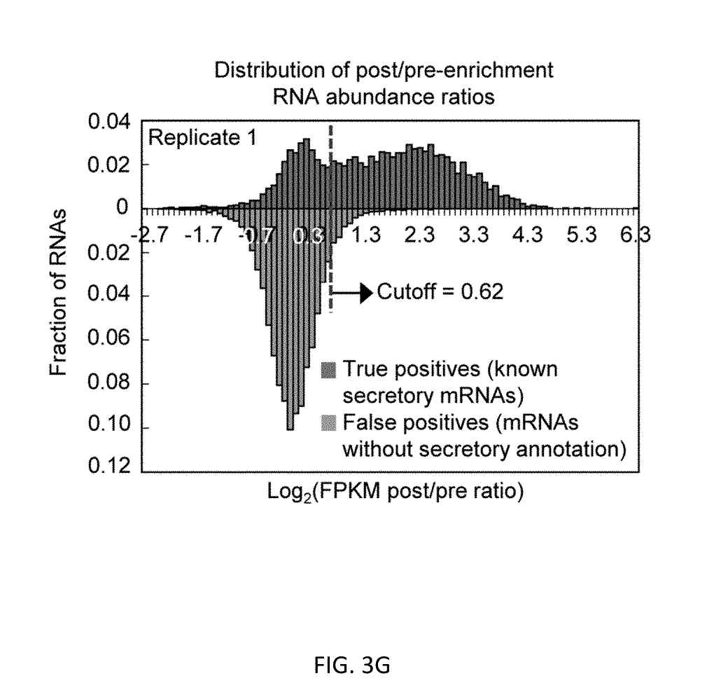

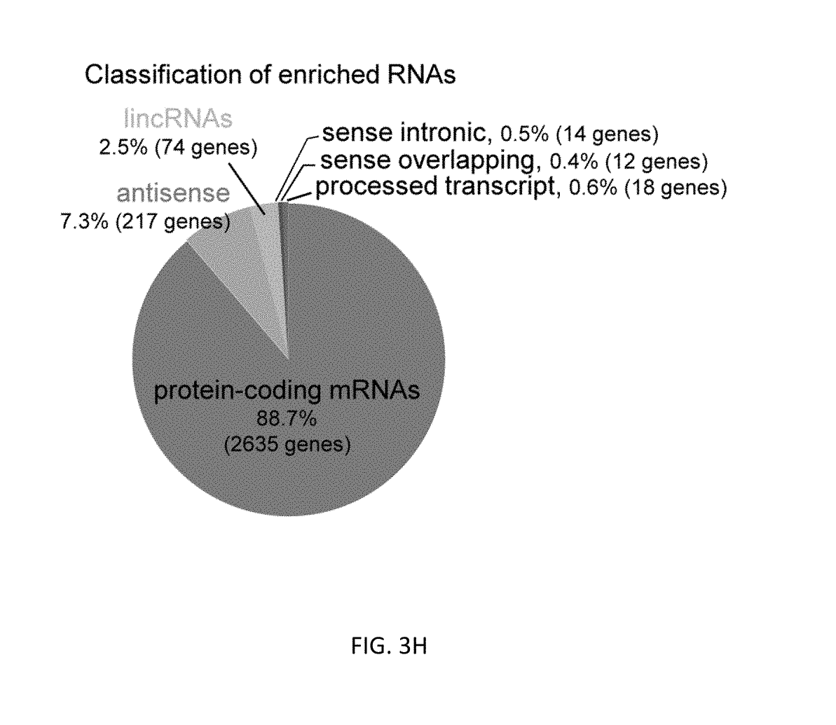

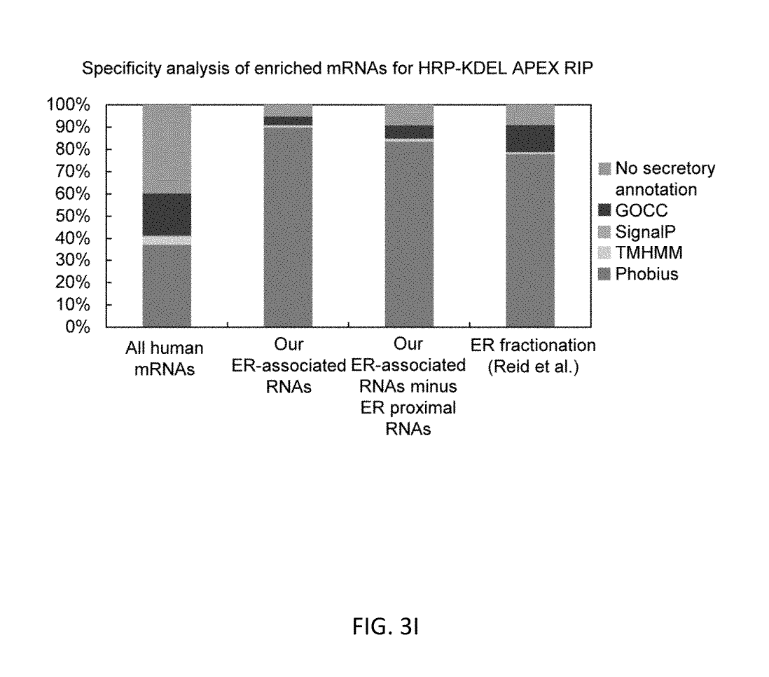

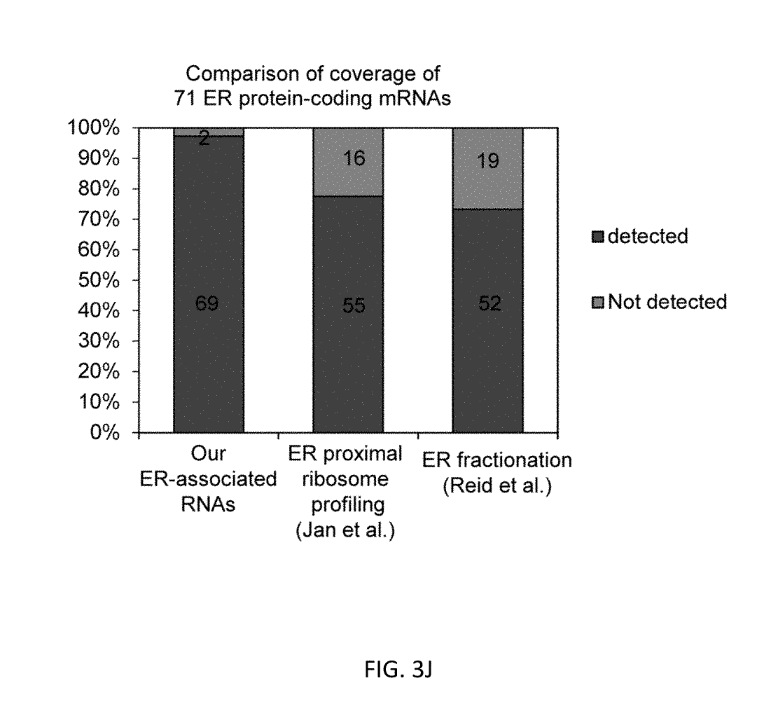

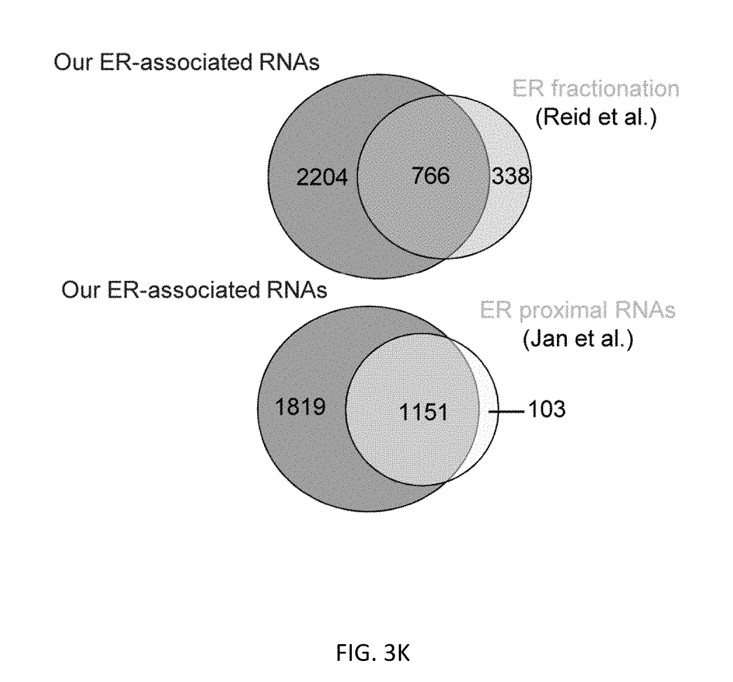

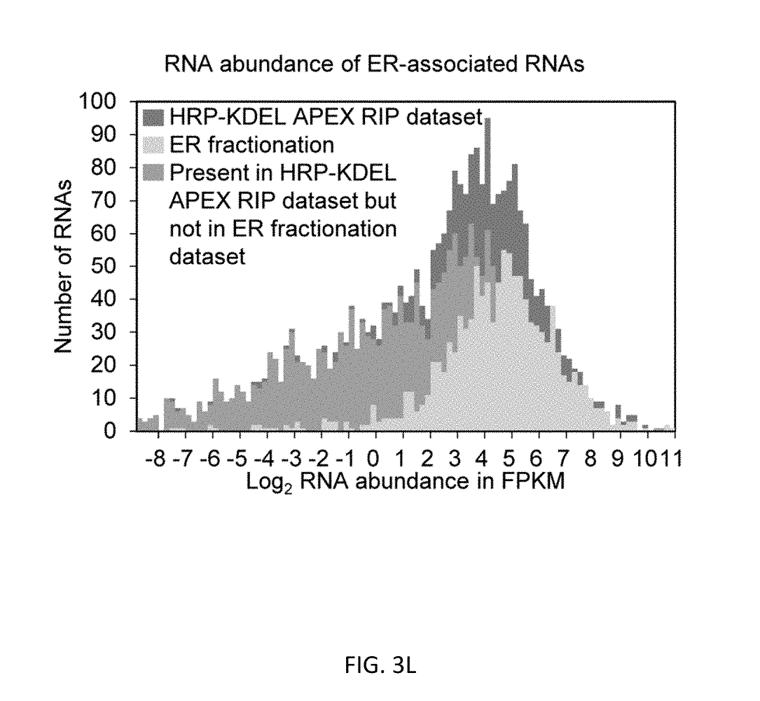

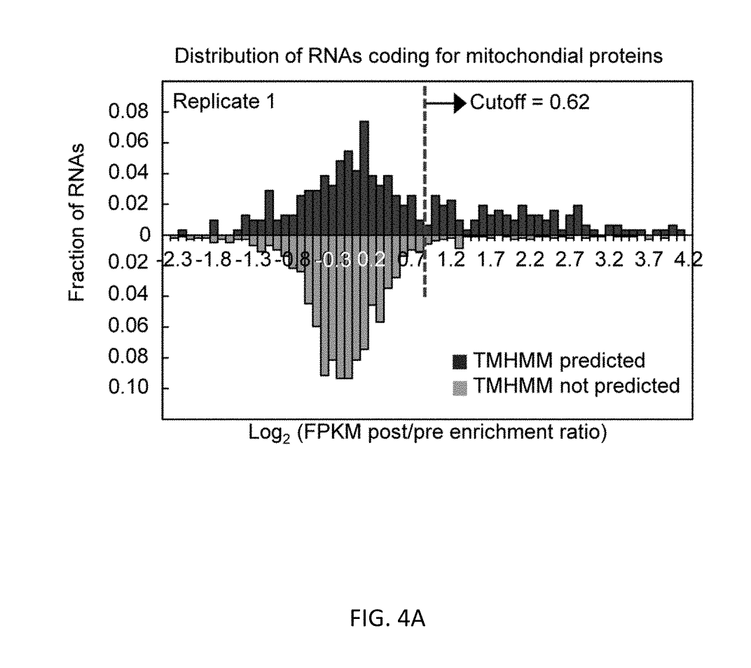

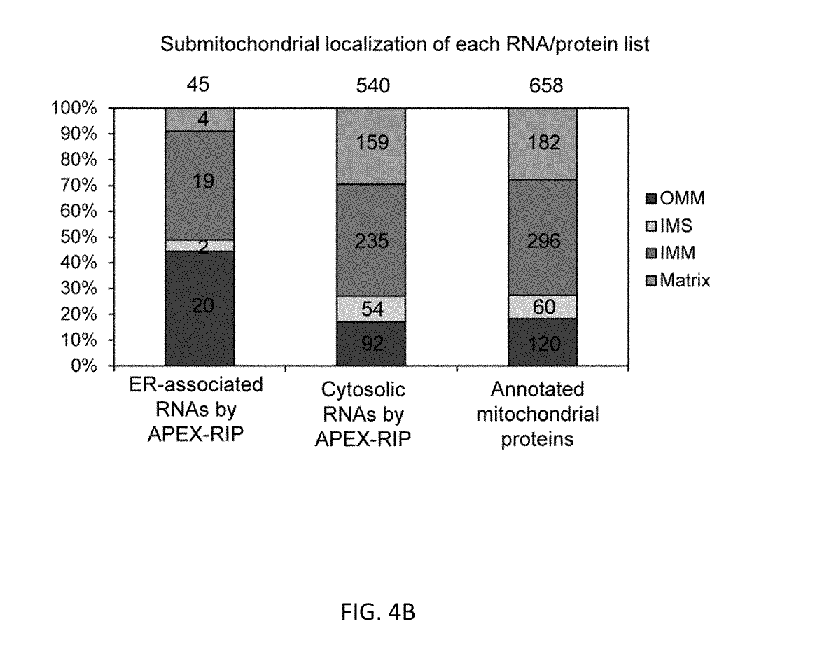

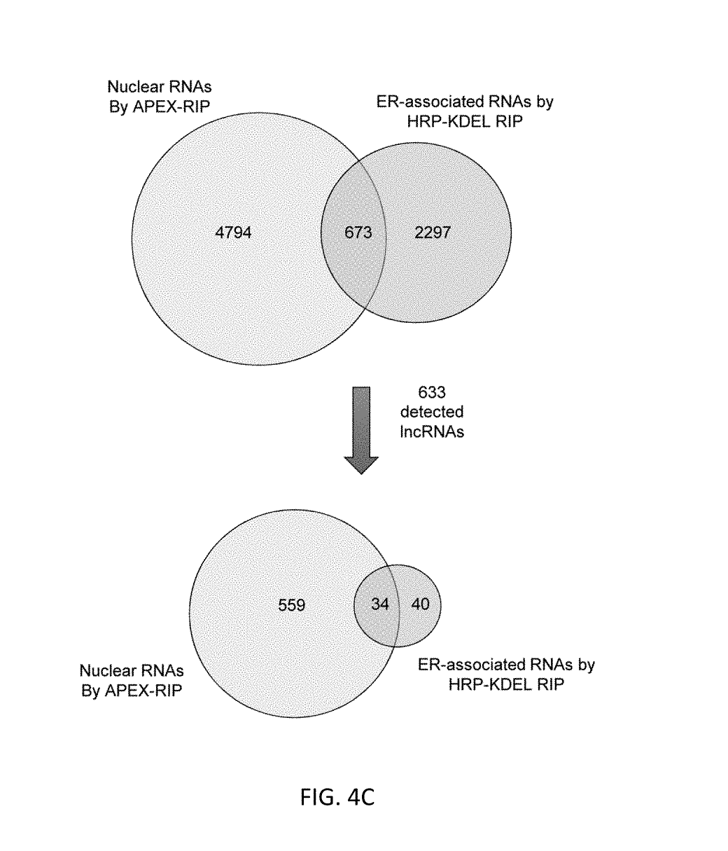

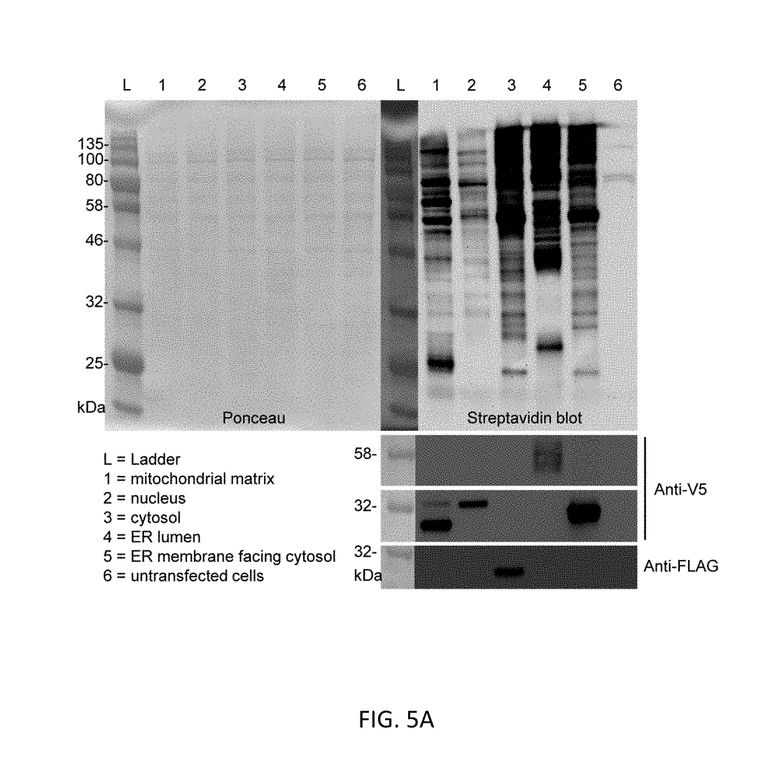

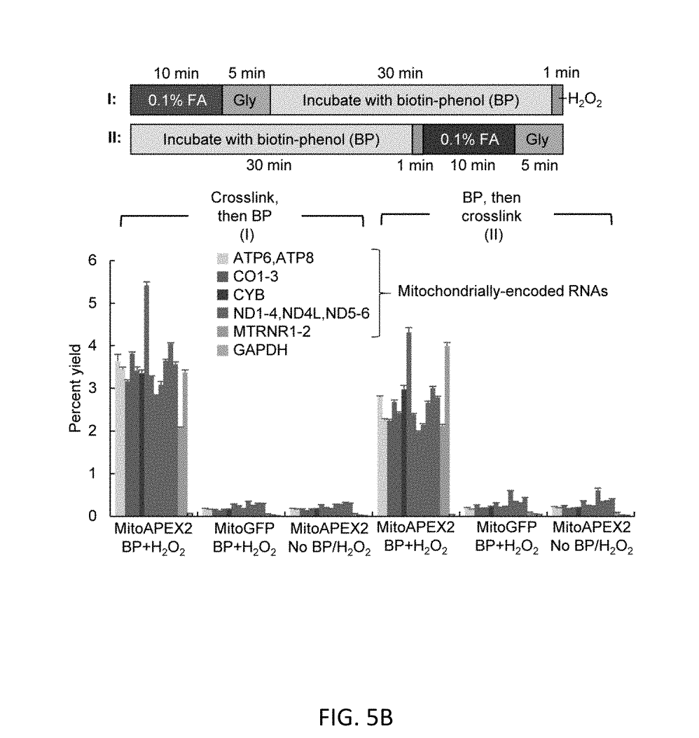

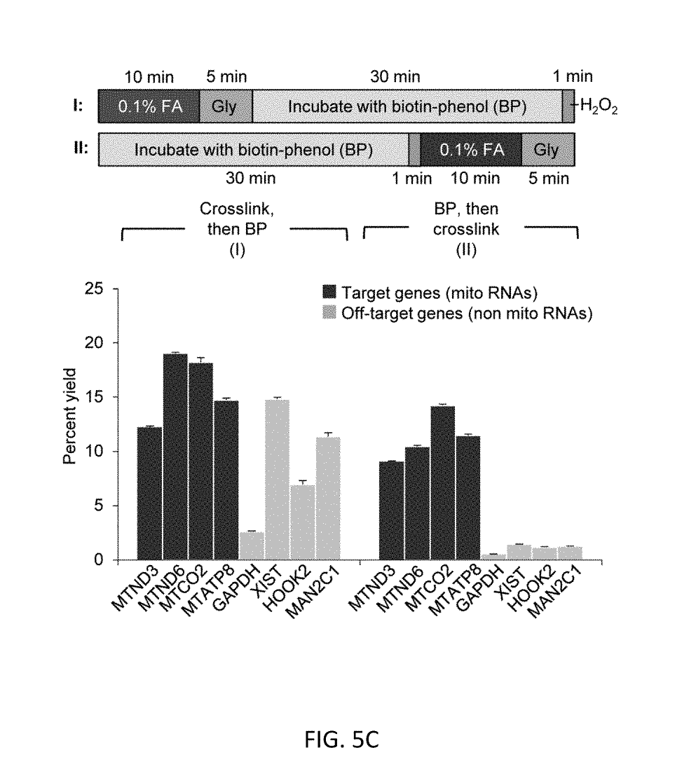

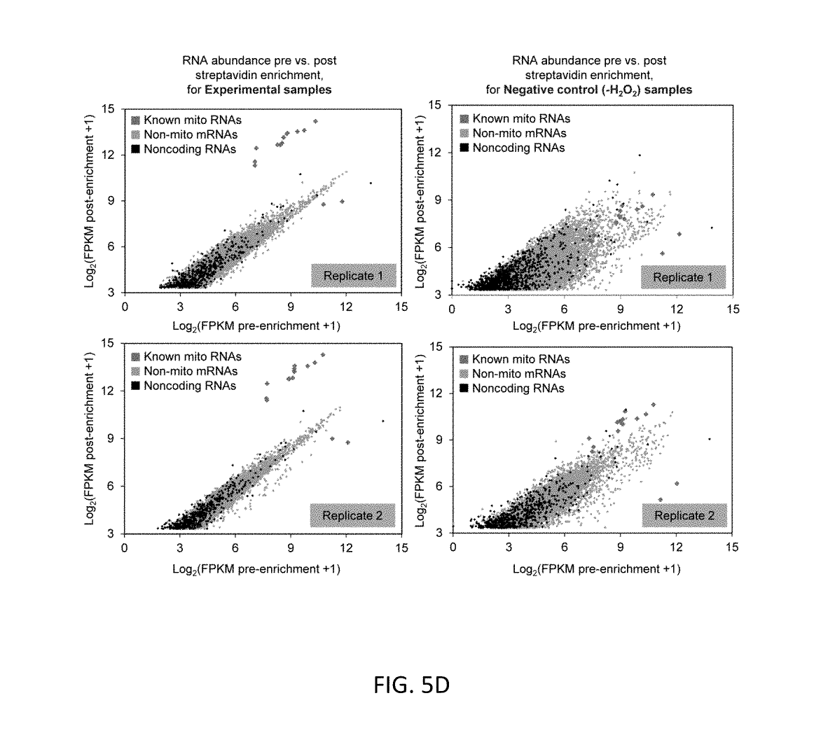

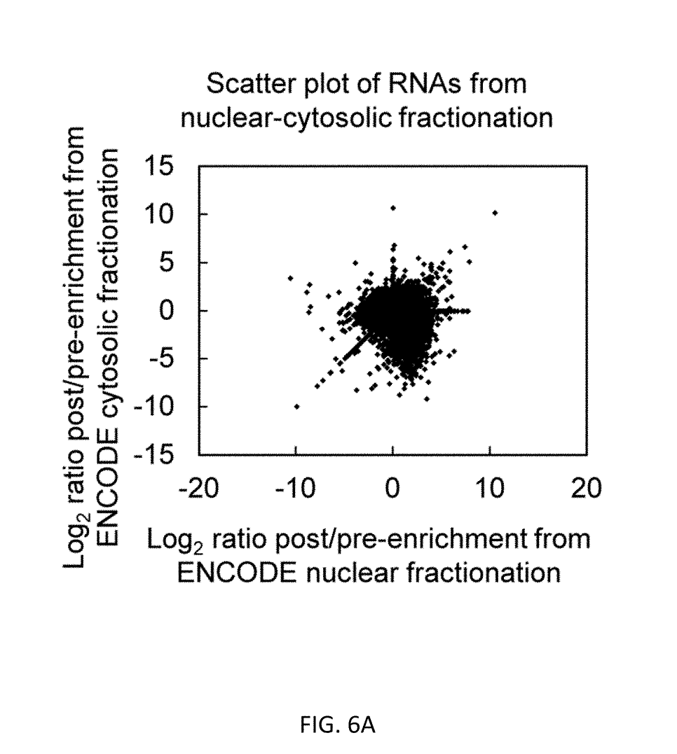

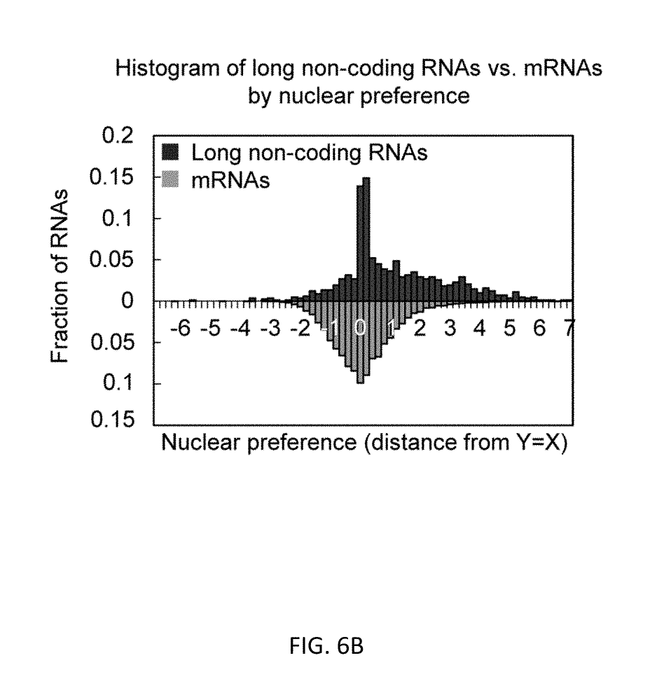

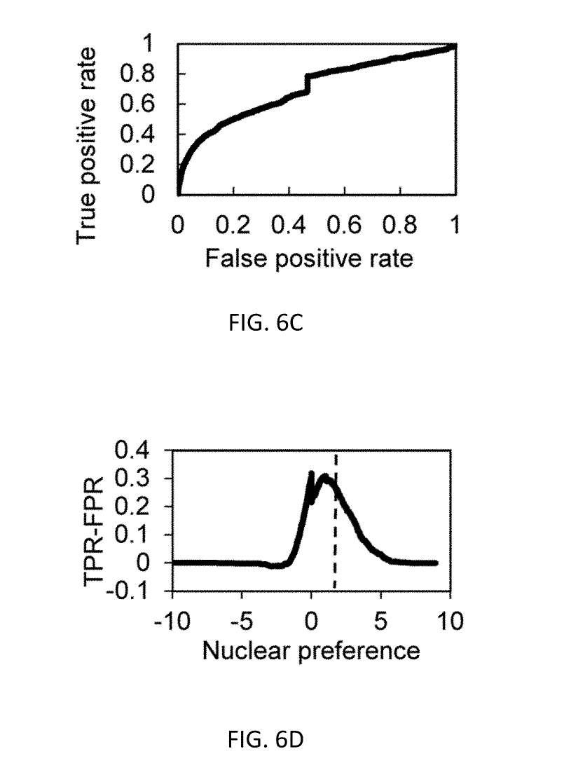

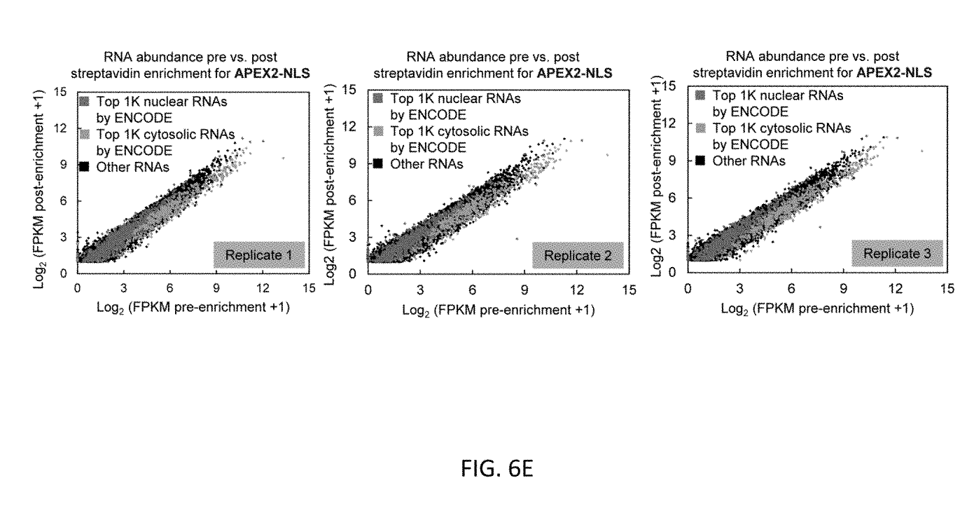

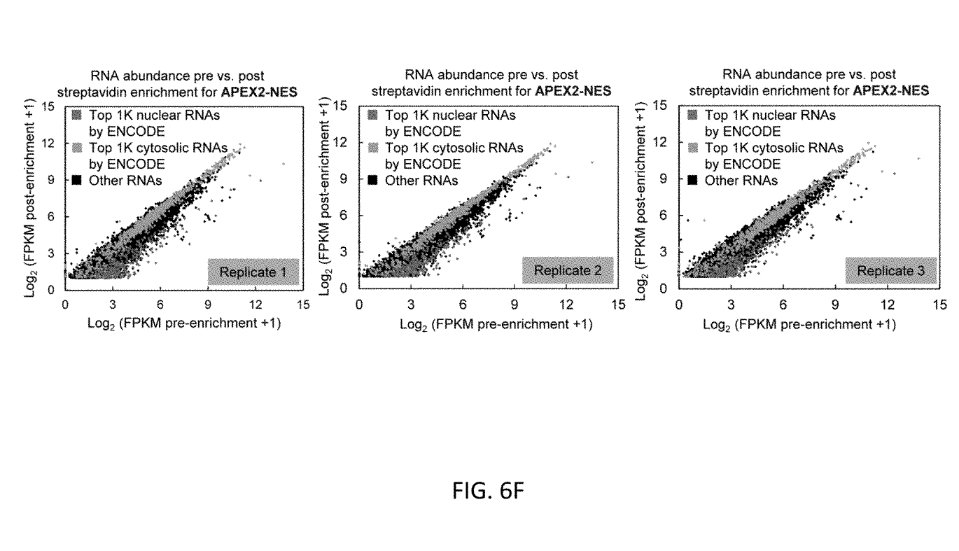

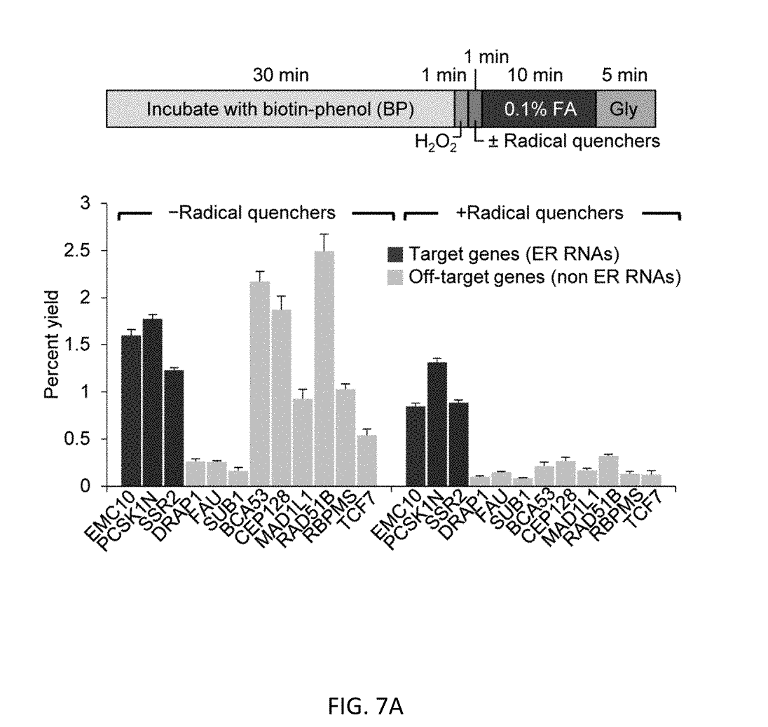

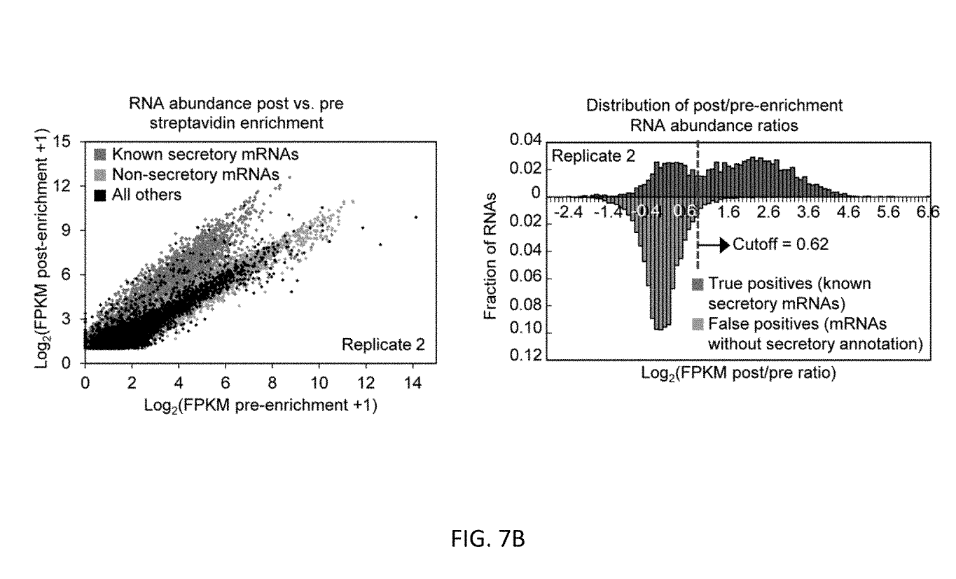

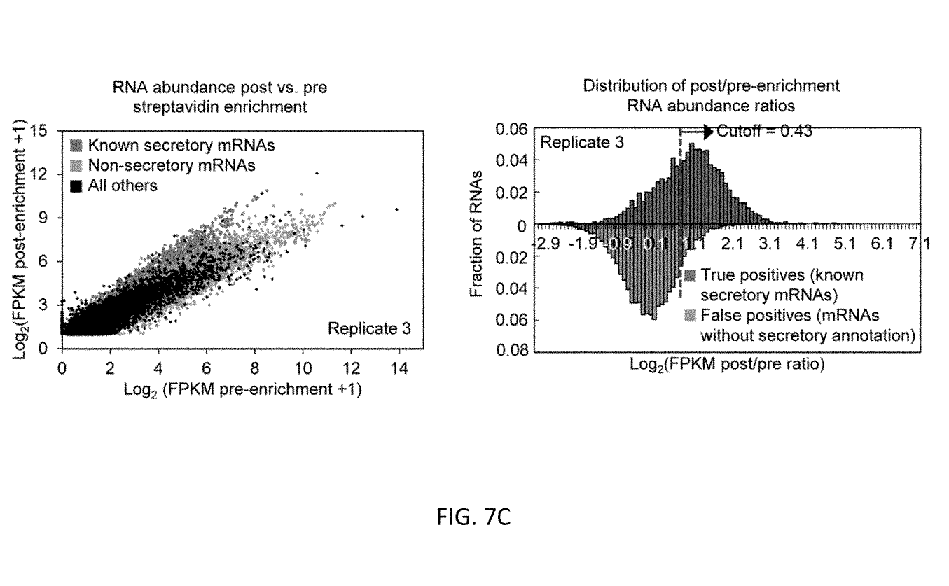

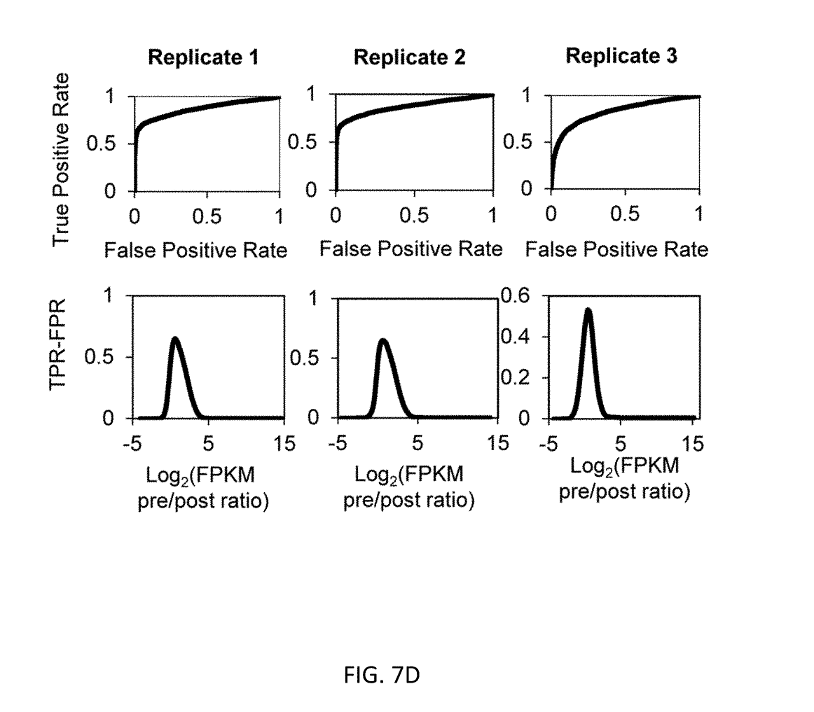

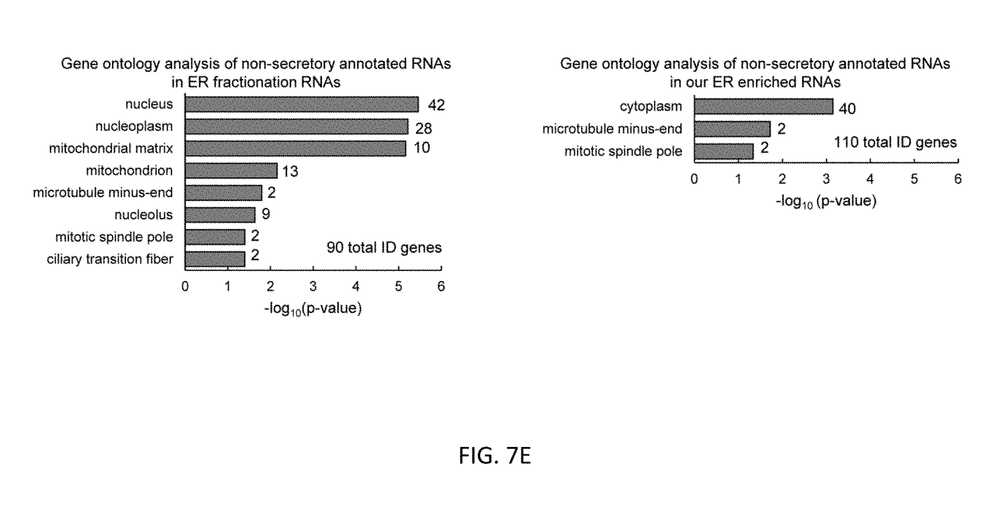

Current methods that identify the location of RNAs en masse have proven cumbersome, low-throughput, difficult and noisy. The technology disclosed here surpasses other methods in accuracy, depth, ease and cost of use. Our method combines proximity-specific biotinylation, crosslinking and RNA deep sequencing (RNA Seq), to identify RNAs within or near a particular subcellular compartment in vivo. First, transgenic cell lines or organisms are generated in which an enzyme capable of proximity-specific biotinylation is targeted to the compartment of interest. Cells or organisms are then briefly treated with this enzyme's substrate(s), inducing pervasive biotinylation of proteins and nucleic acids within the target compartment. Immediately thereafter, covalent crosslinks are generated, linking proteins to nearby RNAs. Hence, all RNA species within or near the target compartment are physically coupled to biotin. Cells are then lysed, biotinylated species are enriched by conventional methods, and bound RNAs are liberated and analyzed by deep sequencing (FIG. 1A).

Most existing technologies for studying RNA localization are either based on microscopic fluorescence imaging, or require native purification of the target subcellular compartment in vitro. Methods in the former category are often extremely low-throughput (i.e. allowing only a handful of RNAs to be analyzed at a time), or alternatively require highly specialized next-generation microscopic equipment and/or a large array of custom biochemical reagents. Methods in the latter category require the development of a robust purification scheme for the target compartment, which may entail substantial loss of loosely affiliated RNAs, or may generally be impossible. In both cases, separating the biological signal from experimental noise can be extremely challenging. In contrast, the method presented here uses standard genetic manipulation techniques and commercially available reagents to provide an exquisitely sensitive, broad and unbiased view of subcellular RNA localization.

Since all biological processes-including development and disease-fundamentally depend on both RNA function and cellular organization, we anticipate that this technology will enable a vast array of insights with potential clinical relevance. Identifying RNA mislocalization events that contribute to a diseased state may help in identifying new targets for therapeutic development. Likewise, comparing subcellular transcriptomes and proteomes may facilitate the identification of novel ribonucleoprotein (RNP) interactions, which may likewise be therapeutic targets. In a broader sense, characterization of the contributing factors (sequences, structures, binding partners, etc. . . . ) that specify RNA subcellular targeting may allow one to manipulate the localization of endogenous or artificial RNPs, a new avenue for the design of advanced RNA therapeutics.

### Example 2

### APEX-Fusion Constructs

Plasmids and Cloning

APEX-fusion constructs were generated using standard restriction enzyme-based, Gibson assembly, or standard QuikChange methods. All the lentiviral constructs were cloned into plx304 vector. The non-lentiviral constructs are cloned into pCDNA3 plasmid. See Table 1.

TABLE 1
Genetic constructs used in this study

| Name | Features | Promoter/Vector | Details |
|---|---|---|---|
| Mito-V5-APEX | NotI-mito-BamHI- | CMV/pCDNA3 | Mito is a 24-amino acid |
| | V5-APEX-XhoI | | mitochondrial targeting |
| | | | sequence (MTS) derived |
| | | | from COX4. |
| | | | V5: GKPIPNPLLGLDST |
| | | | (SEQ ID NO: 1) |
| mito-V5- | mito-BamHI-V5- | CMV/pLX304 | |
| APEX2 | APEX2-NheI | | |
| Mito-GFP | NotI-mito-BamHI- | CMV/pCDNA3 | |
| | BFP-XhoI | | |
| V5-APEX2- | NotI-V5-APEX2- | CMV | NLS: DPKKKRKV |
| NLS | EcoRI-3xNLS-NheI | | (SEQ ID NO: 2) |
| FLAG-APEX2- | BstBI-FLAG- | CMV/pLX304 | NES: LWLPPLERLTLD |
| NES | APEX2-NES-XhoI | | (SEQ ID NO: 3) |
| HRP-V5-KDEL | IgK-HRP-V5-KDEL | CMV | IgK is N-terminal |
| | | | signaling sequence that |
| | | | brings protein to ER |
| | | | (METDTLLLWVLLLWVPGSTGD, |
| | | | SEQ ID NO: 4). |
| | | | KDEL is ER-retaining |
| | | | sequence |
| ERM-APEX2-V5 | BstBI-ERM- | CMV/pLX304 | ERM is ER membrane |
| | APEX2-V5-NheI | | targeting sequence |
| | | | derived from N- |
| | | | terminal 27 amino acids |
| | | | of rabbit P450 C1 |
| | | | (MDPVVVLGLCLSCLLLLSLWKQ |
| | | | SYGGG, SEQ ID NO: 5) |

### Example 3

### Mammalian Cell Culture

HEK-293T from ATCC (passages<25) were cultured in a 1:1 DMEM:MEM mixture (Cellgro) supplemented with 10% FBS, 50 units/mL penicillin, and 50 μg/mL streptomycin at 37° C. under 5% CO₂. Mycoplasma testing was not performed before experiments. For fluorescence microscopy imaging experiments, cells were grown on 7×7-mm glass coverslips in 48-well plates. To improve the adherence of HEK-293T cells, we pretreated glass slides with 50 μg/mL fibronectin (Millipore) for 20 minutes at 37° C. before cell plating and washed three times with Dulbecco's phosphate-buffered saline (DPBS), pH 7.4.

### Example 4

### Preparation of Cells Stably Expressing APEX-Fusion Constructs

Human embryonic kidney (HEK) 293T cells were cultured in Minimum Essential Medium (MEM) supplemented with 10% fetal bovine serum, penicillin, and streptomycin at 37° C. under 5% CO₂. To prepare lentivirus, cells were plated on a T25 plate. Each plate of cells was transfected with 2.5 μg of APEX2 fusion plasmid, 0.25 μg VSVG, and 2.25 μg dR8.91 using 10 μl Lipofectamine 2000 (Invitrogen) in MEM (without serum or antibiotics) at ˜70% confluence. VSVG and dR8.91 are lentiviral packaging plasmids (Pagliarini et al., 2008). The cells were transfected for 3 hours. Then, the media was replaced with 2 ml fresh growth media. After 48 hours, the supernatant was collected and filtered through a 0.45 μm syringe filter. The filtered supernatant was used to infect cells immediately. HEK 293T cells were infected at ˜50% confluency, followed by selection with 8 μg/mL blasticidin in growth medium for 7 days before further analysis.

### Example 5

### Biotin Phenol Labeling and Crosslinking

For crosslinking followed by labeling, the cells were plated in 6-well plate. At 90% confluency, the cells were washed once with PBS, followed by 0.1% formaldehyde in PBS for 10 minutes. The crosslinking was quenched by spiking in glycine (final concentration 125 mM). After washing three times with PBS, the cells were incubated with 500 μM BP in PBS at room temperature. After 30 minutes, H₂O₂ was spiked in (final concentration 1 mM) for 1 minute. Then the BP solution was removed and washed twice with quenchers (final concentration 5 mM Trolox, 10 mM Ascorbate, 10 mM sodium azide). The cells were scrapped and pelleted for further analysis.

For labeling first followed by crosslinking, the cells were plated in 6-well plate. At 90% confluency, the media was replaced with 500 μM BP in cell culture media. The cells were incubated for 30 minutes at 37° C. Then H₂O₂ was spiked in. After 1 minute, the media was replaced with PBS+10 mM ascorbate and 5 mM Trolox for 1 minute, followed by 1 minutes incubation of PBS+0.1% formaldehyde, 10 mM ascorbate, and 5 mM Trolox. Then, the media was replaced and incubated with fresh PBS+0.1% formaldehyde, 10 mM ascorbate, and 5 mM Trolox for 9 minutes. For labeling in mitochondrial matrix, after 1 minute of H₂O₂, the cells were washed with PBS+0.1% formaldehyde, 10 mM ascorbate, and 5 mM Trolox 1 minute twice, and 8 minutes for the last wash. After the last incubation with formaldehyde, glycine was spiked in (final concentration 125 mM) for 5 minutes. Then the cells were washed twice with PBS+10 mM ascorbate and 5 mM Trolox. After washing, The cells were scrapped and pelleted for further analysis.

### Example 6

### Streptavidin Bead Enrichment of Biotinylated Material and RNA Isolation

The labeled cell pellet was lysed in 1 mL RIPA buffer for 5 min at 4° C. and further sonicated three times of 30 seconds at 10% amplitude with 0.7 seconds on and 1.3 seconds off on ice. The lysates were cleared by centrifugation at 15,000 g for 5 minutes at 4° C. The lysate was diluted by 1 mL Native lysis buffer (NLB: 25 mM Tris, 1.5 M KCl, 0.5% NP-40 pH 7.5). Streptavidin-coated magnetic beads (Pierce) were washed twice with 1:1 RIPA: NLB buffer, and 80% of each sample was separately incubated with 50 μL of magnetic bead slurry with rotation for 2 hours at 4° C. The beads were subsequently washed twice with 1 mL RIPA lysis buffer, once with 1 mL of 1 M KCl, once with 1 mL of 2 M urea in 10 mM Tris-HCl pH 8.0, once with 1 mL RIPA lysis buffer, once with 1 mL of 1:1 of RIPA: NLB buffer, once with NLB buffer, and once with TE buffer. The materials were released from the beads by incubating with 2 mg/mL proteinase K (Ambion), 2% lauryl sarcoside, 10 mM EDTA, 1% RNaseOUT, 5 mM DTT in 100 μL PBS at 42° C. for 1 hour and 55 C for 1 hour. The released RNAs were cleaned up by AMPure XP magnetic beads according to manufacture protocol. After cleanup, the DNA residues were digested by DNase I at 37° C. for 30 minutes. The RNAs were cleaned up again by AMPure XP magnetic beads.

### Example 7

### Real Time-PCR Assay

Whole cell RNAs (no enrichment) and enriched RNAs were reverse transcribed using SuperScript III Reverse Transcriptase kit (ThermoFisher Scientific) with random hexamers (ThermoFisher Scientific). The relative quantity of cDNA was measured using SYBR Green PCR master mix (Applied Biosystems) according to manufacturer's protocol. qRT-PCR primer sequences are listed in Table 2. All data were acquired by Applied Biosystems 7900HT Fast real time PCR instrument and the data was analyzed by Real time PCR Miner website.

TABLE 2
qRT-PCR primers used in this study

| Primer/probe name | Sequence (5′-3′) |
|---|---|
| MT-ND1 forward | CACCTCTAGCCTAGCCGTTT |
| | (SEQ ID NO: 6) |
| MT-ND1 reverse | CCGATCAGGGCGTAGTTTGA |
| | (SEQ ID NO: 7) |
| MT-ND2 forward | CTTAAACTCCAGCACCACGAC |
| | (SEQ ID NO: 8) |
| MT-ND2 reverse | AGCTTGTTTCAGGTGCGAGA |
| | (SEQ ID NO: 9) |
| MT-ND3 forward | CCGCGTCCCTTTCTCCATAA |
| | (SEQ ID NO: 10) |
| MT-ND3 reverse | AGGGCTCATGGTAGGGGTAA |
| | (SEQ ID NO: 11) |
| MT-ND4 forward | ACAACACAATGGGGCTCACT |
| | (SEQ ID NO: 12) |
| MT-ND4 reverse | CCGGTAATGATGTCGGGGTT |
| | (SEQ ID NO: 13) |
| MT-ND4L forward | TCGCTCACACCTCATATCCTC |
| | (SEQ ID NO: 14) |
| MT-ND4L reverse | AGGCGGCAAAGACTAGTATGG |
| | (SEQ ID NO: 15) |
| MT-ND5 forward | TCCATTGTCGCATCCACCTT |
| | (SEQ ID NO: 16) |
| MT-ND5 reverse | GGTTGTTTGGGTTGTGGCTC |
| | (SEQ ID NO: 17) |
| MT-ND6 forward | GGGTTGAGGTCTTGGTGAGT |
| | (SEQ ID NO: 18) |
| MT-ND6 reverse | ACCAATCCTACCTCCATCGC |
| | (SEQ ID NO: 19) |
| MT-CYTB forward | TCTTGCACGAAACGGGATCA |
| | (SEQ ID NO: 20) |
| MT-CYTB reverse | CGAGGGCGTCTTTGATTGTG |
| | (SEQ ID NO: 21) |
| MT-COX1 forward | TCCTTATTCGAGCCGAGCTG |
| | (SEQ ID NO: 22) |
| MT-COX1 reverse | ACAAATGCATGGGCTGTGAC |
| | (SEQ ID NO: 23) |
| MT-COX2 forward | AACCAAACCACTTTCACCGC |
| | (SEQ ID NO: 24) |
| MT-COX2 reverse | CGATGGGCATGAAACTGTGG |
| | (SEQ ID NO: 25) |
| MT-COX3 forward | CTAATGACCTCCGGCCTAGC |
| | (SEQ ID NO: 26) |
| MT-COX3 reverse | AGGCCTAGTATGAGGAGCGT |
| | (SEQ ID NO: 27) |
| MT-ATP6 forward | TTCGCTTCATTCATTGCCCC |
| | (SEQ ID NO: 28) |
| MT-ATP6 reverse | GGGTGGTGATTAGTCGGTTGT |
| | (SEQ ID NO: 29) |
| MT-ATP8 forward | ACTACCACCTACCTCCCTCAC |
| | (SEQ ID NO: 30) |
| MT-ATP8 reverse | GGCAATGAATGAAGCGAACAGA |
| | (SEQ ID NO: 31) |
| MT-RNR1 forward | CATCCCCGTTCCAGTGAGTT |
| | (SEQ ID NO: 32) |
| MT-RNR1 reverse | TGGCTAGGCTAAGCGTTTTGA |
| | (SEQ ID NO: 33) |
| MT-RNR2 forward | CAGCCGCTATTAAAGGTTCGT |
| | (SEQ ID NO: 34) |
| MT-RNR2 reverse | AAGGCGCTTTGTGAAGTAGG |
| | (SEQ ID NO: 35) |
| GAPDH forward | TTCGACAGTCAGCCGCATCTTCTT |
| | (SEQ ID NO: 36) |
| GAPDH reverse | GCCCAATACGACCAAATCCGTTGA |
| | (SEQ ID NO: 37) |
| XIST forward | CCCTACTAGCTCCTCGGACA |
| | (SEQ ID NO: 38) |
| XIST reverse | ACACATGCAGCGTGGTATCT |
| | (SEQ ID NO: 39) |
| EMC10 forward | TTCATTGAGCGCCTGGAGAT |
| | (SEQ ID NO: 40) |
| EMC10 reverse | TTCATTGAGCGCCTGGAGAT |
| | (SEQ ID NO: 41) |
| PCSK1N forward | GAGACACCCGACGTGGAC |
| | (SEQ ID NO: 42) |
| PCSK1N reverse | AATCCGTCCCAGCAAGTACC |
| | (SEQ ID NO: 43) |
| SSR2 forward | GTTTGGGATGCCAACGATGAG |
| | (SEQ ID NO: 44) |
| SSR2 reverse | CTCCACGGCGTATCTGTTCA |
| | (SEQ ID NO: 45) |
| TMX1 forward | ACGGACGAGAACTGGAGAGA |
| | (SEQ ID NO: 46) |
| TMX1 reverse | ATTTTGACAAGCAGGGCACC |
| | (SEQ ID NO: 47) |
| SFT2D2 forward | CCATCTTCCTCATGGGACCAG |
| | (SEQ ID NO: 48) |
| SFT2D2 reverse | GCAGAACACAGGGTAAGTGC |
| | (SEQ ID NO: 49) |
| EPT1 forward | TGGCTTTCTGCTGGTCGTAT |
| | (SEQ ID NO: 50) |
| EPT1 reverse | AATCCAAACCCAGTCAGGCA |
| | (SEQ ID NO: 51) |
| DRAP1 forward | ACATCCCACCTGAAGCAGTG |
| | (SEQ ID NO: 52) |
| DRAP1 reverse | GATGCCACCAGGTCCTTCAA |
| | (SEQ ID NO: 53) |
| FAU forward | TCCTAAGGTGGCCAAACAGG |
| | (SEQ ID NO: 54) |
| FAU reverse | GTGGGCACAACGTTGACAAA |
| | (SEQ ID NO: 55) |
| SUB1 forward | CGTCACTTCCGGTTCTCTGT |
| | (SEQ ID NO: 56) |
| SUB1 reverse | TGATTTAGGCATCGCTTCGC |
| | (SEQ ID NO: 57) |
| LSM6 forward | CGGACGACCAGTTGTGGTAA |
| | (SEQ ID NO: 58) |
| LSM6 reverse | CCAGGACCCCTCGATAATCC |
| | (SEQ ID NO: 59) |
| COPS2 forward | AGGAGGACTACGACCTGGAAT |
| | (SEQ ID NO: 60) |
| COPS2 reverse | GCCGCTTTTGGGTCATCTTC |
| | (SEQ ID NO: 61) |
| CGGBP1 forward | GCCTCGTCCACTTTCCCTAA |
| | (SEQ ID NO: 62) |
| CGGBP1 reverse | TCATGCCTTTACGTAGGATCGAG |
| | (SEQ ID NO: 63) |
| BCA53 forward | TCTTGCCTGCTCCACAGTTT |
| | (SEQ ID NO: 64) |
| BCA53 reverse | CAAACACCAAGGAGGGGTCT |
| | (SEQ ID NO: 65) |
| CEP128 forward | TACAGTAATGGACAGGCGGG |
| | (SEQ ID NO: 66) |
| CEP128 reverse | TCCGGAGTTGGTCGATTGAT |
| | (SEQ ID NO: 67) |
| MAD1L1 forward | CGAGTCTGCCATCGTCCAA |
| | (SEQ ID NO: 68) |
| MAD1L1 reverse | GCACTCTCCACCTGCTTCTT |
| | (SEQ ID NO: 69) |
| RAD51B forward | TTTGGACGAAGCCCTGCAT |
| | (SEQ ID NO: 70) |
| RAD51B reverse | CACAACCTGGTGGACCTGTA |
| | (SEQ ID NO: 71) |
| RBPMS forward | ACAGTCGCTCAGAAGCAGAG |
| | (SEQ ID NO: 72) |
| RBPMS reverse | CGAAGCGGATGCCATTCAAA |
| | (SEQ ID NO: 73) |
| TCF7 forward | TCAACAGCCCACATCCCAC |
| | (SEQ ID NO: 74) |
| TCF7 reverse | AGAGGCCTGTGAACTTGCTT |
| | (SEQ ID NO: 75) |

### Example 8

### Library Preparation and Sequencing Analysis

The RNAs were ribosome-depleted using Ribo-Zero Gold rRNA removal kit. The library was prepared using the TruSeq RNA sample preparation kit, v2 (Illumina) as described in manufacture protocol. The indexed libraries were pooled together and sequenced by Illumina HiSeq 2500. For characterization of gene expression, sequencing reads were mapped to a custom gene set comprising UCSC known human genes (hg19) using TopHat2 with default options. Differential analysis of gene expression was assessed using Cuffdiff2 with default options.

### Example 9

### Transfection and Immunofluorescence Staining

To transfect the plasmids, cells plated on 7×7-mm glass coverslips in 48-well plates were transfected at ˜50-60% confluency with 150 ng of the corresponding plasmids and 1 μL of Lipofectamine 2000 for 3 hours. 24 hours after transfection, cell were fixed with 4% paraformaldehyde in PBS at room temperature for 10 minutes. Cells were then washed with PBS three times and permeabilized with cold methanol at −20° C. for 5 minutes. Cells were washed again three times with PBS. Cells were then incubated with primary antibodies in 1% BSA in PBS for 1 hour at room temperature. After washing three times with PBS, cells were incubated with secondary antibodies in 1% BSA in PBS for 30 minutes. Cells were then washed three times with PBS and imaged by confocal microscope.

### Example 10

### Gels and Western Blots

HEK 293T cells stably expressing the indicated constructs were plated in 6-well plates. After labeling, the cells were scraped and pelleted by centrifugation at 3,000 g for 10 minutes. The pellet was stored at −80° C. and then lysed with RIPA lysis buffer (50 mM Tris, 150 mM NaCl, 0.1% SDS, 0.5% sodium deoxycholate, 1% Triton X-100, 1× protease cocktail (Sigma Aldrich), 1 mM PMSF (phenylmethylsulfonyl fluoride), for 5 min at 4° C. The cell pellet was resuspended by gentle pipetting. Lysates were clarified by centrifugation at 15,000 g for 10 minutes at 4° C. before separation on a SDS-PAGE gel. Gels were transferred to nitrocellulose membrane, stained by Ponceau S (10 minutes in 0.1% (w/v) Ponceau S in 5% acetic acid/water). The blots were then blocked and stained with primary and secondary antibodies.

While the preferred embodiments of the invention have been illustrated and described, it will be appreciated that various changes can be made therein without departing from the spirit and scope of the invention.

What is claimed is:

1. A method of mapping subcellular localization of nucleic acids in a cell, the method comprising:
a) introducing a tagging enzyme into the cell, wherein the tagging enzyme is targeted to a subcellular region of interest;
b) contacting the cell with a tagging substrate for the tagging enzyme, wherein the tagging enzyme catalyzes a reaction with the tagging substrate resulting in covalent attachment of a tag to proteins within an intracellular spatial location around the tagging enzyme; and
c) contacting the cell with a crosslinking agent before or after step (b), wherein the crosslinking agent covalently couples the proteins to nearby nucleic acids to produce protein-nucleic acid fusions;
d) isolating the tagged protein-nucleic acid fusions using an agent that selectively binds to the tag; and
e) analyzing the tagged protein-nucleic acid fusions to produce a map of the subcellular localization of the nucleic acids.

2. The method of claim 1, wherein the nucleic acids are RNA or DNA.

3. The method of claim 1, wherein the tagging enzyme is a peroxidase.

4. The method of claim 3, wherein the peroxidase is a horseradish peroxidase or an ascorbate peroxidase.

5. The method of claim 4, wherein the ascorbate peroxidase is APEX or APEX2.

6. The method of claim 3, further comprising contacting the cell with hydrogen peroxide.

7. The method of claim 4, wherein the tagging substrate is biotin-phenol or a derivative thereof.

8. The method of claim 7, wherein the tagging substrate is O-acetylated biotin-phenol.

9. The method of claim 7, wherein said tagging of the proteins comprises reaction of the biotin-phenol or derivative thereof with the hydrogen peroxide to produce a biotin-phenoxyl radical that reacts with nearby proteins resulting in biotinylation of said proteins.

10. The method of claim 1, wherein the tagging enzyme is a biotin ligase.

11. The method of claim 10, wherein the biotin ligase is BirA.

12. The method of claim 10, further comprising contacting the cell with biotin.

13. The method of claim 1, wherein the tag is biotin and biotinylated protein-nucleic acid fusions are isolated by binding to a biotin-binding protein.

14. The method of claim 13, wherein the biotin-binding protein is streptavidin or avidin.

15. The method of claim 1, further comprising treating the cell with a radical quencher after said tagging of the proteins.

16. The method of claim 15, wherein the radical quencher is ascorbate or 6-hydroxy-2,5,7,8-tetramethylchroman-2-carboxylic acid (TROLOX).

17. The method of claim 1, wherein the tagging enzyme comprises a targeting sequence that directs the tagging enzyme to the subcellular region of interest.

18. The method of claim 17, wherein the targeting sequence is selected from the group consisting of a secretory protein signal sequence, a membrane protein signal sequence, a nuclear localization sequence, a mitochondrial localization sequence, an outer mitochondrial membrane sequence, an endoplasmic reticulum localization sequence, an endoplasmic reticulum membrane targeting sequence, a nucleolar localization signal sequence, a nuclear export signal sequence, a peroxisome localization sequence, and a protein binding motif sequence.

19. The method of claim 17, wherein the targeting sequence comprises a sequence selected from the group consisting of SEQ ID NOS:1-5.

20. The method of claim 1, wherein the tagging enzyme is covalently linked to a peptide or protein that directs the tagging enzyme to the subcellular region of interest.

21. The method of claim 20, wherein the protein is a cytosolic protein, a nuclear protein, a membrane protein, a mitochondrial protein, a P-body protein, or a secretory pathway protein.

22. The method of claim 1, wherein said introducing the tagging enzyme into the cell comprises transfecting the cell with a recombinant polynucleotide comprising a promoter operably linked to a polynucleotide encoding the tagging enzyme.

23. The method of claim 22, wherein the recombinant polynucleotide comprises a plasmid or viral vector.

24. The method of claim 23, wherein the viral vector is a lentivirus vector.

25. The method of claim 1, wherein the crosslinking agent is formaldehyde, glutaraldehyde, dimethyl suberimidate, N-hydroxysuccinimide, ultraviolet light, a crosslinking agent comprising an adiazomethane, diazoacetyl, or carbodiimide functional group, or a click chemistry crosslinking agent comprising an azide or alkyne functional group.

26. The method of claim 25, wherein the tagging substrate is a phenol derivative comprising an alkyne or azide functional group suitable for crosslinking by click chemistry.

27. The method of claim 1, further comprising identifying at least one ribonucleoprotein (RNP) interaction.

28. The method of claim 1, further comprising sequencing at least one RNA or DNA molecule in the tagged protein-nucleic acid fusions.

29. The method of claim 1, further comprising multiplex sequencing of the tagged protein-nucleic acid fusions.

30. The method of claim 29, wherein said sequencing comprises performing deep sequencing or next-generation sequencing.

31. The method of claim 1, further comprising calculating the frequencies of one or more RNA molecules that are present within the intracellular spatial location or quantitating one or more RNA molecules that are present within the intracellular spatial location.

32. The method of claim 1, further comprising identifying at least one RNA or DNA molecule in the tagged protein-nucleic acid fusions.

33. The method of claim 32, wherein said at least one RNA is selected from the group consisting of a messenger RNA, a ribosomal RNA, a transfer RNA, a non-coding RNA, and a regulatory RNA.

34. The method of claim 1, wherein the cell is exposed to a test condition prior to said contacting the cell with the tagging substrate or the crosslinking agent.

35. The method of claim 34, wherein the test condition comprises exposing the cell to a drug, a ligand for a receptor, a hormone, a second messenger, a pathogen, or a genetic modification.

36. The method of claim 35, wherein the genetic modification comprises introduction of a vector, short hairpin RNA (shRNA), small interfering RNA (siRNA), microRNA (miRNA), or CRISPR-associated system into the cell.

37. The method of claim 34, wherein the test condition comprises exposing the cell to a change in temperature, growth media, membrane potential, or osmotic pressure.

38. The method of claim 34, wherein a map of the subcellular localization of the RNA molecules within the intracellular spatial location is compared to a reference map for a cell that is not exposed to the test condition.

39. The method of claim 1, wherein a map of the subcellular localization of the nucleic acid molecules within the intracellular spatial location is compared to a reference map for a cell at a different developmental stage.

40. The method of claim 1, wherein the cell is a eukaryotic cell, a prokaryotic cell, or an archaeon cell.

41. The method of claim 40, wherein the cell is an animal cell, plant cell, fungal cell, or protist cell.

42. The method of claim 1, wherein the nucleic acids are RNA selected from the group consisting of animal RNA, bacterial RNA, fungal RNA, protist RNA, plant RNA, and viral RNA.

43. The method of claim 1, wherein the cell is an artificial cell encapsulating the nucleic acids.

44. The method of claim 43, wherein the artificial cell comprises a nanoparticle, liposome, polymersome, or microcapsule.

45. The method of claim 1, wherein the cell is a human cell.

46. The method of claim 1, further comprising amplifying at least one RNA.

47. The method of claim 46, wherein said amplifying comprises performing reverse transcription polymerase chain reaction (RT-PCR).

48. The method of claim 1, further comprising lysing the cell.

49. The method of claim 1, wherein the agent that selectively binds to the tag is selected from the group consisting of an antibody, a probe, a ligand, or an aptamer.

50. The method of claim 49, wherein the agent is immobilized on a solid support.
